(19) United States
(12) Patent Application Publication
(10) Pub. No.: US 2022/0289705 A1
(43) Pub. Date: Sep. 15, 2022

(54) PROCESS FOR THE PREPARATION OF RIDINILAZOLE AND CRYSTALLINE FORMS THEREOF

(71) Applicant: Summit (Oxford) Limited, Abingdon (GB)

(72) Inventors: Francis X. Wilson, Welwyn Garden City, Hertsforshire (GB); Nigel ADAMS, Welwyn Garden City, Hertsforshire (GB); Jean-Francois CARNIAUX, Marcham, Abingdon (GB)

(21) Appl. No.: 17/627,076

(22) PCT Filed: Jul. 16, 2020
(86) PCT No.: PCT/GB2020/051710

§ 371 Date: Jan. 13, 2022

(30) Foreign Application Priority Data
Jul. 17, 2019 (GB) 1910250.8
Aug. 23, 2019 (GB) 1912144.1

(51) Int. Cl. C07D 401/04

(52) U.S. Cl. CPC C07D 401/04; C07B 2200/13

(57) ABSTRACT

Described are processes for the preparation of 2,2′-di(pyridin-4-yl)-1H,1′H-5,5′-bibenzo[d]imidazole (which may also be known as 5,5′bis-[2-(4-pyridinyl)-1H-benzimidazole]), referenced herein by the INN name ridinilazole, and pharmaceutically acceptable derivatives, salts, hydrates, solvates, complexes, bioisosteres, metabolites or prodrugs thereof. The invention also relates to various compositions of purified ridinilazole, to various crystalline forms of ridinilazole, to processes for their preparation and to related pharmaceutical preparations and uses thereof (including their medical use and their use in the efficient large-scale synthesis of ridinilazole).

### 1. FIELD OF THE INVENTION

The present invention relates to processes for the preparation of 2,2′-di(pyridin-4-yl)-1H,1H-5,5′-bibenzo[d]imidazole (which may also be known as 5,5′-bis[2-(4-pyridinyl)-1H-benzimidazole], 2,2′-bis(4-pyridyl)-3H,3′H-5,5′-bibenzimidazole or 2-pyridin-4-yl-6-(2-pyridin-4-yl-3H-benzimidazol-5-yl)-1H-benzimidazole), referenced herein by the INN name ridinilazole, and pharmaceutically acceptable derivatives, salts, hydrates, solvates, complexes, bioisosteres, metabolites or prodrugs thereof. The invention also relates to various crystalline forms of ridinilazole, to processes for their preparation and to related pharmaceutical preparations and uses thereof (including their medical use and their use in the efficient large-scale synthesis of ridinilazole).

### 2. BACKGROUND OF THE INVENTION

Infection with Clostridioides difficile (previously named Clostridium difficile) (CDI) causes Clostridioides difficile-associated diseases (CDAD). Over 450,000 cases of CDI occur in the US annually, with over 80,000 first recurrences and approximately 29,000 deaths. The most common precipitant is antibiotic use. Antibiotics cause loss of colonization resistance with the potential establishment of a long-lasting, species-poor microbiota susceptible to pathogen invasion. Oral vancomycin and metronidazole treatment are associated with high CDI recurrence rates, likely due to deleterious effects on resident colonic flora. Recurrences are costly in terms of both clinical burden and healthcare resource utilization. In one study, readmission was required in approximately one-third of recurrence cases.

Both microbiota biomass and composition at the intestinal-bacterial interface likely influence the C. difficile colonization niche. Although colonization resistance has been associated with specific taxa, it is likely that different, yet diverse, microbiota community structures can confer protection. Consistent characteristics of communities susceptible to CDI are low diversity levels and diminished metabolic function with loss of relative abundance of members of the Bacteroidetes and Firmicutes phyla and increases in that of Proteobacteria. Faecal microbiota transplantation (FMT) normalizes these features and breaks the CDI recurrence cycle.

In aggregate, these data support a role for CDI agents with minimal effects on indigenous microbiota to reduce risk of recurrence.

Ridinilazole (also known as SMT19969, and which may be variously referenced as 2,2′-di(pyridin-4-yl)-1H,1′H-5,5′-bibenzo[d]imidazole or 5,5′-bis[2-(4-pyridinyl)-1H-benzimidazole] in the literature), is a narrow-spectrum, poorly-absorbable, potent C. difficile-targeting antimicrobial. Ridinilazole may be represented by the following formula:

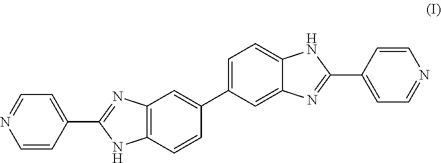 [SMILES: c1cc(-c2nc3cc(-c4ccc5[nH]c(-c6ccncc6)nc5c4)ccc3[nH]2)ccn1]

In a recent Phase 2 randomized, controlled, double-blinded clinical trial comparing its efficacy to vancomycin, ridinilazole was associated with marked reduction in rates of recurrent disease (14.3% vs. 34.8%). Ridinilazole exhibits enhanced preservation of the human intestinal microbiota compared to vancomycin (which may contribute to the reduced CDI recurrence observed in the Phase 2 study).

Accordingly, there is need for an efficient synthesis of ridinilazole.

Control of genotoxic and potentially genotoxic impurities (PGIs) during drug manufacture is of great concern and acceptable levels must be no higher than that justified by safety data.

There is a need in the art for processes by which drug candidates can be prepared which allow effective removal of PGIs.

The present inventors have now developed efficient processes for producing ridinilazole, as well as its pharmaceutically acceptable salts, hydrates, solvates, complexes, bioisosteres, metabolites or prodrugs, which: (a) are suitable for large-scale synthesis under GMP conditions; and (b) reduce PGIs to levels acceptable for commercial production of drug formulations.

The present inventors have now also discovered three distinct crystalline forms (polymorphs) of ridinilazole which have particular utility in the above processes and which find application in the efficient large-scale synthesis of ridinilazole for medicinal use (as well as in medicine more generally).

WO2010/063996 describes various benzimidazoles, including ridinilazole, and their use as antibacterials (including in the treatment of CDAD).

WO 2011/151621 describes various benzimidazoles and their use as antibacterials (including in the treatment of CDAD).

WO2007056330, WO2003105846 and WO2002060879 disclose various 2-amino benzimidazoles as antibacterial agents.

WO2007148093 discloses various 2-amino benzothiazoles as antibacterial agents.

WO2006076009, WO2004041209 and Bowser et al. (Bioorg. Med. Chem. Lett., 2007, 17, 5652-5655) disclose various substituted benzimidazole compounds useful as anti-infectives that decrease resistance, virulence, or growth of microbes. The compounds are said not to exhibit intrinsic antimicrobial activity in vitro.

U.S. Pat. No. 5,824,698 discloses various dibenzimidazoles as broad-spectrum antibiotics, disclosing activity against both Gram-negative and Gram-positive bacteria, including Staphylococcus spp. and Enterococcus spp. However, this document does not disclose activity against anaerobic spore-forming bacteria and in particular does not disclose activity against any Clostridioides spp. (including C. difficile).

US 2007/0112048 A1 discloses various bi- and triarylimidazolidines and bi- and triarylamidines as broad-spectrum antibiotics, disclosing activity against both Gram-negative and Gram-positive bacteria, including Staphylococcus spp., Enterococcus spp. and Clostridioides spp. However, this document does not disclose compounds of formula (I) as described herein.

Chaudhuri et al. (2007) J. Org. Chem. 72, 1912-1923 describe various bis-2-(pyridyl)-1H-benzimidazoles (including compounds of formula I as described herein) as DNA binding agents. This document is silent as to potential antibacterial activity.

Singh et al. (2000) Synthesis 10: 1380-1390 describe a condensation reaction for producing 2,2′-di(pyridin-4-yl)-1H,1′H-5,5′-bibenzo[d]imidazole using 4-pyridine carboxaldehyde, FeCl₃, O₂, in DMF at 120° C.

Bhattacharya and Chaudhuri (2007) Chemistry—An Asian Journal 2: 648-655 describe a condensation reaction for producing 2,2′-di(pyridin-4-yl)-1H,1′H-5,5′-bibenzo[d]imidazole using 4-pyridine carboxaldehyde and nitrobenzene at 120° C.

WO2019/068383 describes the synthesis of ridinilazole by metal-ion catalyzed coupling of 3,4,3′,4′-tetraaminobiphenyl with 4-pyridinecarboxaldehyde in the presence of oxygen, followed by the addition of a complexing agent.

### 4. SUMMARY OF THE INVENTION

According to a first aspect of the invention, there is provided a composition comprising a mixture of compounds, said mixture comprising ridinilazole and compounds of formulae (II) and (IV):

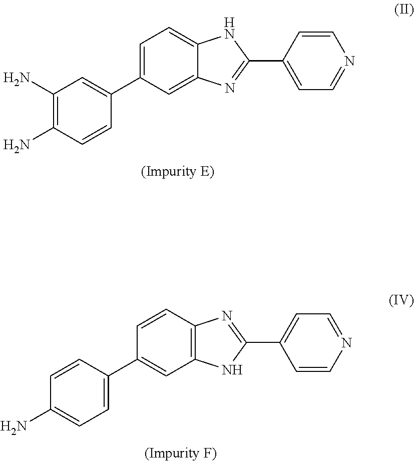 [SMILES: Nc1ccc(-c2ccc3[nH]c(-c4ccncc4)nc3c2)cc1N ; Nc1ccc(-c2ccc3nc(-c4ccncc4)[nH]c3c2)cc1]

wherein the combined amount of Impurities E and F in the mixture is less than 100 ppm.

In preferred embodiments, the ridinilazole is present as a crystalline form of ridinilazole tetrahydrate (Form A) characterized by a powder X-ray diffractogram (XRPD) comprising characteristic peaks at 2-Theta angles of (11.02±0.2)°, (16.53±0.2)° and (13.0±0.2)°.

In a second aspect of the invention, there is provided a process for producing a composition according to the first aspect of the invention comprising the steps of: (a) providing a crude ridinilazole composition comprising a mixture of compounds, said mixture comprising ridinilazole and compounds of formulae (II) and (IV):

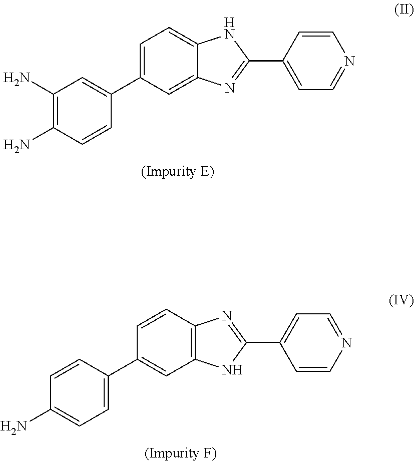 [SMILES: Nc1ccc(-c2ccc3[nH]c(-c4ccncc4)nc3c2)cc1N ; Nc1ccc(-c2ccc3nc(-c4ccncc4)[nH]c3c2)cc1]

wherein the combined amount of the Impurities E and F in the mixture is greater than 100 ppm; and then
(b) removing Impurities E and F from the mixture to produce a purified ridinilazole composition in which the combined amount of Impurities E and F present in the mixture is less than 100 ppm.

In a third aspect, the invention provides a composition according to the first aspect of the invention which is obtainable (or produced) by the process of the invention.

In another aspect the invention provides a pharmaceutical composition comprising an effective amount of the composition of the invention and a pharmaceutically acceptable excipient.

In another aspect, the invention provides a composition of the invention for use in therapy or prophylaxis.

In another aspect, the invention provides a composition of the invention for use in the therapy or prophylaxis of CDI or CDAD.

In another aspect, the invention provides the use of the composition of the invention for the manufacture of a medicament for the treatment, therapy or prophylaxis of CDI or CDAD.

In another aspect, the invention provides a crystalline form of ridinilazole tetrahydrate (Form A) characterized by a powder X-ray diffractogram comprising characteristic peaks at 2-Theta angles of (11.02±0.2)°, (16.53±0.2)° and (13.0±0.2)°.

In another aspect, the invention provides a crystalline form of ridinilazole anhydrate (Form D) characterized by a powder X-ray diffractogram comprising characteristic peaks at 2-Theta angles of (12.7±0.2)°, (23.18±0.2)° and (27.82±0.2)°, optionally comprising characteristic peaks at 2-Theta angles of (12.7±0.2)°, (23.18±0.2)°, (27.82±0.2)°, (19.5±0.2)° and (22.22±0.2)°.

Other aspects and embodiments of the invention are set out in the claims appended hereto.

### 5. DETAILED DESCRIPTION AND EXAMPLES OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### 5.1 Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The phrase “consisting essentially of” is used herein to require the specified integer(s) or steps as well as those which do not materially affect the character or function of the claimed invention.

As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

The pharmaceutical compositions of the invention may be comprised in a pharmaceutical kit, pack or patient pack.

As used herein, the term “pharmaceutical kit” defines an array of one or more unit doses of a pharmaceutical composition together with dosing means (e.g. measuring device) and/or delivery means (e.g. inhaler or syringe). The unit doses and/or dosing means may optionally all be contained within common outer packaging. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

As used herein, the term ridinilazole is used to define the compound 2,2′-di(pyridin-4-yl)-1H,1′H-5,5′-bibenzo[d]imidazole (which may also be known as 5,5′-bis[2-(4-pyridinyl)-1H-benzimidazole], 2,2′-bis(4-pyridyl)-3H,3′H-5,5′-bibenzimidazole or 2-pyridin-4-yl-6-(2-pyridin-4-yl-3H-benzimidazol-5-yl)-1H-benzimidazole). The term also includes pharmaceutically acceptable derivatives, salts, hydrates, solvates, complexes, bioisosteres, metabolites or prodrugs of ridinilazole, as herein defined.

The term pharmaceutically acceptable derivative as applied to ridinilazole define compounds which are obtained (or obtainable) by chemical derivatization of the parent compounds of the invention. The pharmaceutically acceptable derivatives are therefore suitable for administration to or use in contact with mammalian tissues without undue toxicity, irritation or allergic response (i.e. commensurate with a reasonable benefit/risk ratio). Preferred derivatives are those obtained (or obtainable) by alkylation, esterification or acylation of the parent compounds of the invention. The derivatives may be active per se, or may be inactive until processed in vivo. In the latter case, the derivatives of the invention act as prodrugs. Particularly preferred prodrugs are ester derivatives which are esterified at one or more of the free hydroxyls and which are activated by hydrolysis in vivo. Other preferred prodrugs are covalently bonded compounds which release the active parent drug according to formula (I) after cleavage of the covalent bond(s) in vivo.

The pharmaceutically acceptable derivatives of the invention retain some or all of the activity of the parent compound. In some cases, the activity is increased by derivatization. Derivatization may also augment other biological activities of the compound, for example bioavailability.

The term pharmaceutically acceptable salt as applied to ridinilazole defines any non-toxic organic or inorganic acid addition salt of the free base compound which is suitable for use in contact with mammalian tissues without undue toxicity, irritation, allergic response and which are commensurate with a reasonable benefit/risk ratio. Suitable pharmaceutically acceptable salts are well known in the art. Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid). The compounds of the invention may be converted into (mono- or di-) salts by reaction with a suitable base, for example an alkali metal hydroxide, methoxide, ethoxide or tert-butoxide, or an alkyl lithium, for example selected from NaOH, NaOMe, KOH, KOtBu, LiOH and BuLi, and pharmaceutically acceptable salts of ridinilazole may also be prepared in this way.

These salts and the free base compounds can exist in either a hydrated or a substantially anhydrous form. Crystalline forms of the compounds of the invention are also contemplated and in general the acid addition salts of the compounds of the invention are crystalline materials which are soluble in water and various hydrophilic organic solvents and which in comparison to their free base forms, demonstrate higher melting points and an increased solubility. For example, the sodium salt of ridinilazole is sufficiently soluble in methanol as to permit the methanol solution to be passed over/through activated charcoal.

The term pharmaceutically acceptable solvate as applied to ridinilazole defines any pharmaceutically acceptable solvate form of a specified compound that retains the biological effectiveness of such compound. Examples of solvates include compounds of the invention in combination with water (hydrates), short-chain alcohols (including isopropanol, ethanol and methanol), dimethyl sulfoxide, ethyl acetate, acetic acid, ethanolamine, acetone, dimethylformamide (DMF), dimethylacetamide (DMAc), pyrrolidones (such as N-Methyl-2-pyrrolidone (NMP)), tetrahydrofuran (THF), and ethers (such as tertiarybutylmethylether (TBME)).

Also included are miscible formulations of solvate mixtures such as a compound of the invention in combination with an acetone and ethanol mixture. In a preferred embodiment, the solvate includes a compound of the invention in combination with about 20% ethanol and about 80% acetone. Thus, the structural formulae include compounds having the indicated structure, including the hydrated as well as the non-hydrated forms.

The term pharmaceutically acceptable prodrug as applied to ridinilazole defines any pharmaceutically acceptable compound that may be converted under physiological conditions or by solvolysis to ridinilazole in vivo, to a pharmaceutically acceptable salt of such compound or to a compound that shares at least some of the antibacterial activity of the specified compound (e.g. exhibiting activity against Clostridioides difficile).

The term pharmaceutically acceptable metabolite as applied to ridinilazole defines a pharmacologically active product produced through metabolism in the body of ridinilazole or salt thereof.

Prodrugs and active metabolites of the compounds of the invention may be identified using routine techniques known in the art (see for example, Bertolini et al., J. Med. Chem., 1997, 40, 2011-2016).

The term pharmaceutically acceptable complex as applied to ridinilazole defines compounds or compositions in which the compound of the invention forms a component part. Thus, the complexes of the invention include derivatives in which the compound of the invention is physically associated (e.g. by covalent or non-covalent bonding) to another moiety or moieties. The term therefore includes multimeric forms of the compounds of the invention. Such multimers may be generated by linking or placing multiple copies of a compound of the invention in close proximity to each other (e.g. via a scaffolding or carrier moiety). The term also includes cyclodextrin complexes.

The term bioisostere (or simply isostere) is a term of art used to define drug analogues in which one or more atoms (or groups of atoms) have been substituted with replacement atoms (or groups of atoms) having similar steric and/or electronic features to those atoms which they replace. The substitution of a hydrogen atom or a hydroxyl group with a fluorine atom is a commonly employed bioisosteric replacement. Sila-substitution (C/Si-exchange) is a relatively recent technique for producing isosteres. This approach involves the replacement of one or more specific carbon atoms in a compound with silicon (for a review, see article by Tacke and Zilch in Endeavour, New Series, 1986, 10, 191-197). The sila-substituted isosteres (silicon isosteres) may exhibit improved pharmacological properties, and may for example be better tolerated, have a longer half-life or exhibit increased potency (see for example article by Englebienne in Med. Chem., 2005, 1(3), 215-226). Similarly, replacement of an atom by one of its isotopes, for example hydrogen by deuterium, may also lead to improved pharmacological properties, for example leading to longer half-life (see for example Kushner et al (1999) Can J Physiol Pharmacol. 77(2):79-88). In its broadest aspect, the present invention contemplates all bioisosteres (and specifically, all silicon bioisosteres) of the compounds of the invention.

In its broadest aspect, the present invention contemplates all tautomeric forms, optical isomers, racemic forms and diastereoisomers of the compounds described herein. Those skilled in the art will appreciate that, owing to the asymmetrically substituted carbon atoms present in the compounds of the invention, the compounds may be produced in optically active and racemic forms. If a chiral centre or another form of isomeric centre is present in a compound of the present invention, all forms of such isomer or isomers, including enantiomers and diastereoisomers, are intended to be covered herein. Compounds of the invention containing a chiral centre (or multiple chiral centres) may be used as a racemic mixture, an enantiomerically enriched mixture, or the racemic mixture may be separated using well-known techniques and an individual enantiomer may be used alone. Thus, references to the compounds of the present invention encompass the products as a mixture of diastereoisomers, as individual diastereoisomers, as a mixture of enantiomers as well as in the form of individual enantiomers.

Therefore, the present invention contemplates all optical isomers and racemic forms thereof of the compounds of the invention, and unless indicated otherwise (e.g. by use of dash-wedge structural formulae) the compounds shown herein are intended to encompass all possible optical isomers of the compounds so depicted. In cases where the stereochemical form of the compound is important for pharmaceutical utility, the invention contemplates use of an isolated eutomer.

As used herein, the term condensation reaction, as applied to 3,3′-diaminobenzidine (DAB) to yield ridinilazole and an intermediate co-product of formula (II), indicates a reaction in which two or more reactants yield a single main product with accompanying formation of a small molecule, e.g. water, ammonia, ethanol, acetic acid or hydrogen sulphide. It is therefore used herein as a term of art sensu lato.

The abbreviation “XRPD” stands for X-ray powder diffraction (or when context permits, an X-ray powder diffractogram).

As used herein the term “room temperature” (RD relates to temperatures between 15 and 25° C.

The term “substantially in accordance” with reference to XRPD diffraction patterns means that allowance is made for variability in peak positions and relative intensities of the peaks. The ability to ascertain substantial identities of X-ray diffraction patterns is within the purview of one of ordinary skill in the art. For example, a typical precision of the 2-Theta values is in the range of ±0.2° 2-Theta. Thus, a diffraction peak that usually appears at 14.9° 2-Theta can appear between 14.7° and 15.1° 2-Theta on most X-ray diffractometers under standard conditions. Moreover, variability may also arise from the particular apparatus employed, as well as the degree of crystallinity in the sample, orientation, sample preparation and other factors. XRPD measurements are typically performed at RT, for example at a temperature of 20° C., and preferably also at a relative humidity of 40%.

As used herein, the term “Form A” of ridinilazole refers to the crystalline form of ridinilazole tetrahydrate characterized by a powder X-ray diffractogram comprising characteristic peaks at 2-Theta angles of (11.02±0.2)°, (16.53±0.2)° and (13.0±0.2)°.

As used herein, the term “Form N” of ridinilazole refers to the crystalline form of ridinilazole tetrahydrate characterized by a powder X-ray diffractogram comprising characteristic peaks at 2-Theta angles of (10.82±0.2)°, (13.35±0.2)° and (19.15±0.2)°, optionally comprising characteristic peaks at 2-Theta angles of (10.82±0.2)°, (13.35±0.2)°, (19.15±0.2)°, (8.15±0.2)° and (21.74±0.2)°.

As used herein, the term “Form D” of ridinilazole refers to the crystalline form of ridinilazole anhydrate characterized by a powder X-ray diffractogram comprising characteristic peaks at 2-Theta angles of (12.7±0.2)°, (23.18±0.2)° and (27.82±0.2)°, optionally comprising characteristic peaks at 2-Theta angles of (12.7±0.2)°, (23.18±0.2)°, (27.82±0.2)°, (19.5±0.2)° and (22.22±0.2)°.

One of ordinary skill in the art will appreciate that an XRPD pattern may be obtained with a measurement error that is dependent upon the measurement conditions employed. In particular, it is generally known that intensities in an XRPD pattern may fluctuate depending upon measurement conditions employed. Relative intensities may also vary depending upon experimental conditions and so relative intensities should not be considered to be definitive. Additionally, a measurement error of diffraction angle for a conventional XRPD pattern is typically about 5% or less, and such degree of measurement error should be taken into account when considering stated diffraction angles. It will be appreciated that the various crystalline forms described herein are not limited to the crystalline forms that yield X-ray diffraction patterns completely identical to the X-ray diffraction patterns depicted in the accompanying Figures. Rather, crystalline forms of ridinilazole that provide X-ray diffraction patterns substantially in accordance (as hereinbefore defined) with those shown in the Figures fall within the scope of the present invention.

As used herein, the term “substantially pure” with reference to a particular crystalline (polymorphic) form of ridinilazole is used to define one which includes less than 10%, preferably less than 5%, more preferably less than 3%, most preferably less than 1% by weight of any other physical form of ridinilazole.

As used herein, the term “Impurity E” defines a compound of formula (II):

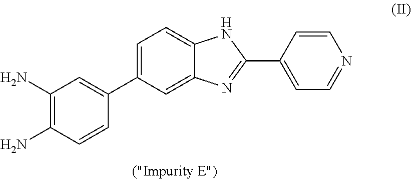 [SMILES: Nc1ccc(-c2ccc3[nH]c(-c4ccncc4)nc3c2)cc1N]

As used herein, the term “Impurity F” defines a compound of formula (IV):

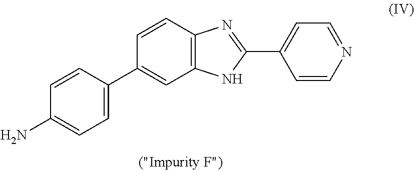 [SMILES: Nc1ccc(-c2ccc3nc(-c4ccncc4)[nH]c3c2)cc1]

### 5.2 Synthesis of Crude Ridinilazole by Imidate-DAB Condensation

The present inventors have determined that a crude ridinilazole composition may be conveniently synthesized by subjecting 3,3′-diaminobenzidine (DAB) to a condensation reaction to yield said ridinilazole. In preferred embodiments, the condensation reaction comprises reacting DAB with an imidate (which may be referenced herein as an “imidate-DAB condensation”). The imidate is preferably methyl isonicotinimidate of formula (V):

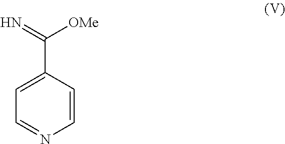

In preferred embodiments, the condensation reaction comprises:
(a) adding sodium methoxide to 4-cyanopyridine to produce the compound of formula (V); and then
(b) reacting the compound of formula (V) of step (a) with said DAB.

The imidate-DAB condensation reaction may comprise two chemical steps:
Step 1a: reaction of 4-cyanopyridine with methanol catalyzed by sodium methoxide to form methyl isonicotimidate; and
Step 1b: coupling of 3,3′-diaminobenzidine (DAB) with methyl isonicotimidate to form crude ridinilazole.

The condensation reaction (step 1 b) may be carried out at a temperature of from 10° C. to 160° C. The reaction may be carried out at the reflux temperature of the solvent at normal pressure (e.g. 152° C. to 154° C. in the case of DMF). The reaction may be carried out in any suitable solvent that does not interfere with the reaction. Suitable solvents include methanol (as in the exemplary reaction scheme 1 shown below). Others include N-methyl-2-pyrrolidone (NMP), dimethylformamide (DMF) and dimethylacetamide (DMAc).

The imidate-DAB condensation may therefore comprise:
(a) adding sodium methoxide to 4-cyanopyridine in methanol to produce the compound of formula (V); and then
(b) adding the compound of formula (V) of step (a) to a mixture of DAB and acetic acid in methanol; or
(c) adding a mixture of DAB and acetic acid in methanol to the compound of formula (V) of step (a).

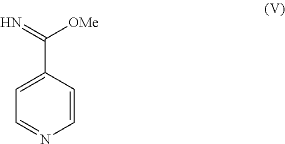

In step 1a, other imidates may be produced and used in the condensation reaction by using different alkoxide/alcohol combinations. For example, sodium ethoxide/ethanol may be used instead of sodium methoxide/methanol, while other cations (preferably alkali metals, such as lithium or potassium) may replace sodium.

In step 1b, the amount of acetic acid is preferably <3.5 equivalents, for example 2.5-3.0 equivalents. Other acids (such as TFA) may be used instead of acetic acid.

There is broad scope for manipulation of the precise conditions of the imidate-DAB condensation reaction and all such manipulations are within the scope of the invention. Resources that would be of help to the skilled person when performing the invention include Vogel's Textbook 5 of Practical Organic Chemistry, Fifth Edition, B. S. Furniss et al, Pearson Education Limited, 1988, which discusses general practical procedure. In addition, methods of synthesis are discussed in Comprehensive Heterocyclic Chemistry, Vol. 1 (Eds.: A R Katritzky, C W Rees), Pergamon Press, Oxford, 1984 and Comprehensive Heterocyclic Chemistry II: A Review of the Literature 1982-1995 The Structure, Reactions, 10 Synthesis, and Uses of Heterocyclic Compounds, Alan R. Katritzky (Editor), Charles W. Rees (Editor), E. F. V. Scriven (Editor), Pergamon Pr, June 1996. Other general resources which would aid the skilled person include March's Advanced Organic Chemistry: Reactions, Mechanisms, and Structure, Wiley-Interscience; 5th edition (Jan. 15, 2001).

A preferred imidate-DAB reaction is shown schematically below:

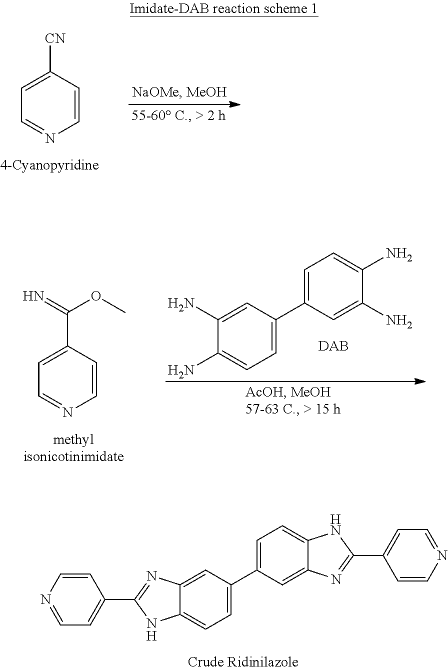

In the exemplary reaction scheme 1 shown above, the condensation reaction starts with the DAB slurry in methanol, and with about three quarters of the imidate feed, the reaction mixture becomes a solution for a short period of time and then the crude ridinilazole product crashes out of solution (and may be recovered as a wet filter cake).

The inventors have found that the dynamics of this crystallization process are capricious, and depend inter alia on stochastic nucleation events. Without wishing to be bound by any theory, it is believed that Impurities E and F become entrained within the ridinilazole crystals (and/or within amorphous regions thereof). For example, it is believed that during the precipitation process, some unreacted Impurity E is trapped in the product crystals and is not able to further react with the imidate (even though the imidate may be present in large excess).

The recovered crude ridinilazole product therefore comprises a mixture of Impurities E and F together with anhydrous crystalline Form D of ridinilazole characterized by an XRPD pattern substantially in accordance with FIG. 3.

### 5.3 Impurities E and F in Crude Ridinilazole

In the synthesis of crude ridinilazole described in Section 5.2 (above), the reaction of DAB with imidate requires two equivalents of the imidate for reaction completion. The inventors have discovered that an intermediate impurity, a compound of formula (II) (herein also referenced as “Impurity E”), is formed when DAB reacts with only one equivalent of the imidate, as shown below:

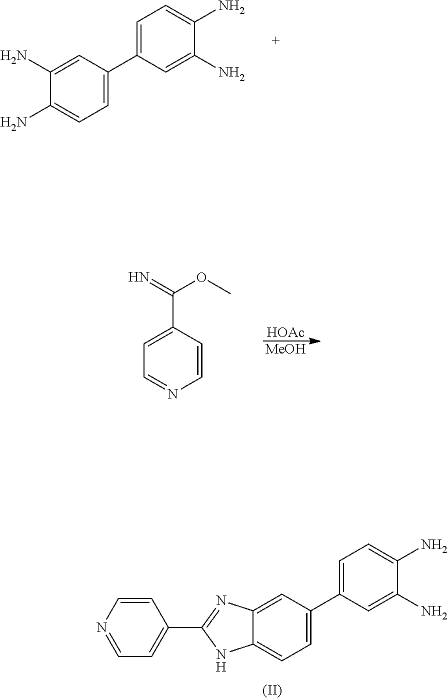

The inventors have also discovered that monoaminobenzidine (MAB, which may be referenced herein as the compound of Formula (III)) is present as an impurity in commercial sources of DAB. They have found that MAB also reacts with the imidate to form a second (process) impurity, this being a compound of formula (IV) (herein also referenced as “Impurity F”), as shown below:

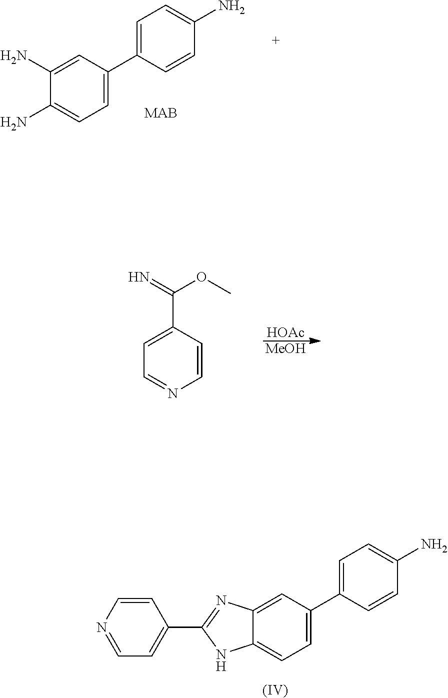

Thus, the crude ridinilazole product produced as described above comprises a mixture of compounds, said mixture comprising ridinilazole and compounds of formulae (II) and (IV) (Impurities E and F, respectively):

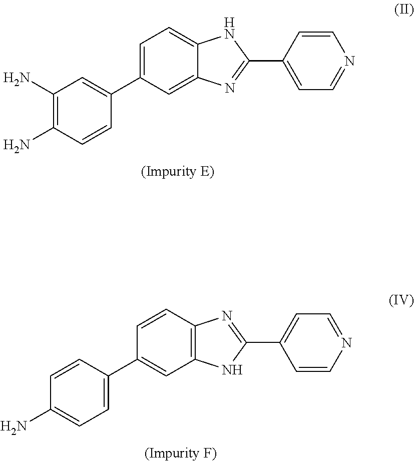 [SMILES: Nc1ccc(-c2ccc3[nH]c(-c4ccncc4)nc3c2)cc1N ; Nc1ccc(-c2ccc3nc(-c4ccncc4)[nH]c3c2)cc1]

The present inventors have surprisingly found that despite the use of the highly toxic DAB and the generation of the Impurities E and F (both of the compounds of formulae (II) and (IV) are potentially genotoxic impurities (PGIs)), efficient large scale GMP synthesis of ridinilazole suitable for use in the formulation of pharmaceutical compositions for dosing at levels for the treatment of CDI and CDAD in humans can be achieved by ensuring that the combined amount of Impurities E and F is less than 100 ppm, as described in more detail below.

Thus, the invention provides a composition comprising a mixture of compounds, said mixture comprising ridinilazole and compounds of formulae (II) and (IV):

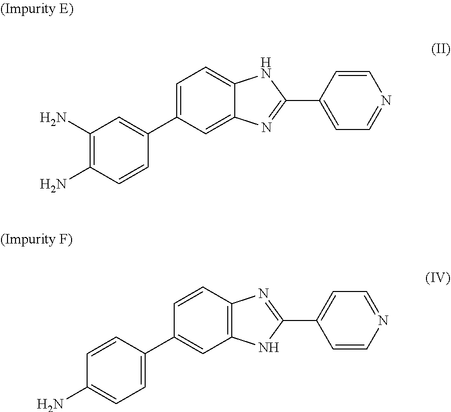 [SMILES: Nc1ccc(-c2ccc3[nH]c(-c4ccncc4)nc3c2)cc1N ; Nc1ccc(-c2ccc3nc(-c4ccncc4)[nH]c3c2)cc1]

wherein the combined amount of Impurities E and F in the mixture is less than 100 ppm.

In preferred embodiments, the ridinilazole is present as a crystalline form of ridinilazole tetrahydrate (Form A) characterized by a powder X-ray diffractogram (XRPD) comprising characteristic peaks at 2-Theta angles of (11.02±0.2)°, (16.53±0.2)° and (13.0±0.2)°.

### 5.4 Determination of Impurities E and F by HPLC-MS

### Materials

Water, Ultra High Quality (e.g. MilliQ) or equivalent

### Formic Acid, 99% for MS

### Methanesulfonic Acid (MSA), 99% Extra Pure

### Methanol, HPLC Grade

### Impurity E

### Impurity F

### Apparatus

Balance: Minimum 5-place balance

### System Parameters

### HPLC/MS System: Agilent LC1200, MSD 61508

### Column: ACE 3 C18, 100×4.6 mm, Cat #ACE-111-1046

Mobile phase A: 0.1% v/v formic acid in DI-water
Mobile phase B: 0.1% v/v formic acid in methanol
Diluent: 98:2:2 v/v/v (Water:MeOH:Methanesulfonic acid)
Injection volume: 2 μL

### Detection: MSD/SIM

m/z 302.1 for Impurity E
m/z 287.1 for impurity F

### Column Temperature: 45° C.

Flow Rate: 1.0 mL/min

### Autosampler T: 5° C.

Needle wash: Diluent

### Gradient

| Time | % A | % B |
|---|---|---|
| 0 | 98 | 2 |
| 2 | 98 | 2 |
| 4 | 70 | 30 |
| 7 | 45 | 55 |
| 10 | 5 | 95 |
| 12 | 5 | 90 |

Post run: 3 minutes
MSD parameters

### Spray Chamber Setting

Drying gas flow 12.0 L/min
Nebulizer pressure 60 psi
Dry gas temperature 350° C.
Capillary voltage 3000V

### MSD Signal Settings

### Ion Source API-ES Positive

### Fragmentor 70

### Gain 1.0

### SIM ion for Impurity E m/z 302.1 at 5.00 min

### SIM ion for Impurity F m/z 287.1 at 5.00 min

### Injector Program

| Function | Parameters |
|---|---|
| Draw | Default volume from sample |
| Wash | Wash needle in flush part for 10 sec |
| Inject | Inject |
| Wait | Wait 10 min |
| Valve | Switch valve to “Bypass” |
| Wait | Wait 0.5 min |
| Valve | Switch valve to “Mainpass” |
| Valve | Switch valve to “Bypass” |
| Wait | Wait 1 min |
| Valve | Switch valve to “Mainpass” |

### Preparation of Solutions

| | |
|---|---|
| Diluent | Mix 980 mL DI-water, 20 mL methanol and 20 mL |
| | Methane sulfonic acid. Mix well on stirring plate. |
| Mobile Phase A | Add 1.0 mL formic acid into 1000 mL DI-water. Mix well. |
| Mobile Phase B | Add 1.0 mL formic acid into 1000 mL methanol. Mix well. |
| Stock standard solutions | Accurately weigh 2 ± 0.2 mg of Impurity E and Impurity F |
| 0.1 mg/mL | reference standards into a 20 mL amber vial. Add 20.00 mL |
| | diluent and vortex to dissolve. |
| | Storage: Stock Standard Solution is stable for 3 days |
| | at 5° C. when stored in amber glassware. |
| Working standard solution | Accurately transfer 100 μL Impurity E and Impurity F |
| 50 ppm Impurity E | Stock Standard Solution into a 20 mL amber volumetric |
| 50 ppm Impurity F | flask. |
| | Dilute to the line with diluent. Vortex to mix. |
| | Storage: Working standard solution needs to be freshly |
| | prepared before injection. |
| Sample Solution (single | Prepare 10 mg/ml solution. Accurately weigh 200 mg |
| preparation) 10 mg/ml | sample and dissolve in 20.0 mL diluent. Sonicate for 5-10 |
| | minutes and vortex to mix. |
| | Storage: Sample solution needs to be freshly prepared |
| | before injection. |

### Injection Sequence

| Solutions | Number of injections |
|---|---|
| Blank solutions | At least 2 |
| Working standard solution | 1 |
| Sample solution | 1 |

### Components:

SMT 19969 Impurity E: Retention time approximately 6.1 minutes
SMT 19969 Impurity F: Retention time approximately 6.6 minutes

### System Suitability

Signal to noise ratio of each impurity peak in the working standard solution must be >10. RT window is within ±1 minute of the expected RT of each component as listed above.

### 5.5 Removal of Impurities E and F from Crude Ridinilazole

By reference to the various dosage regimes indicated for the treatment of CDI or CDAD in human patients, the inventors have determined that the crude ridinilazole product of the above-described process is advantageously further purified to the extent that the combined amount of the compounds of formulae (II) and (IV) (i.e. Impurities E and F, respectively) present in the mixture is less than 100 ppm.

Any suitable purification method, or combination of methods, may be employed, provided that it yields a purified ridinilazole composition in which the combined amount of Impurities E and F present in the mixture is less than 100 ppm.

The invention therefore provides a process for producing a composition comprising a mixture of compounds, said mixture comprising ridinilazole and Impurities E and F, wherein the combined amount of Impurities E and F in the mixture is less than 100 ppm and wherein the process comprises the steps of:
(a) providing a crude ridinilazole composition comprising a mixture of compounds, said mixture comprising ridinilazole and compounds of formulae (II) and (IV):

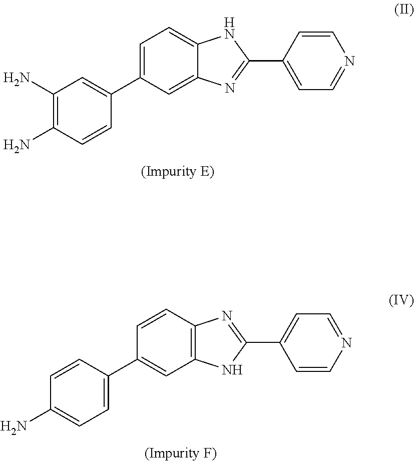 [SMILES: Nc1ccc(-c2ccc3[nH]c(-c4ccncc4)nc3c2)cc1N ; Nc1ccc(-c2ccc3nc(-c4ccncc4)[nH]c3c2)cc1]

wherein the combined amount of the Impurities E and F in the mixture is greater than 100 ppm; and then
(b) removing Impurities E and F from the mixture to produce a purified ridinilazole composition in which the combined amount of Impurities E and F present in the mixture is less than 100 ppm.

It will be appreciated that the purification method(s) described herein may also serve to remove, or reduce the concentration of, other impurities, for example those present in the starting materials, reactants and process reagents (such as DAB and MAB), as well as other process impurities that may arise.

Preferred purification methods for use as the removing step (b), which may be used alone or in any combination, are described in more detail below:

5.5.1 Treatment of Crude Ridinilazole with Imidate to Purge Impurity E

The crude ridinilazole product of the imidate-DAB condensation reaction described in Section 5.2 (above) may be treated with an imidate solution to react with Impurity E and thereby purge it from the mixture.

For example, an imidate solution was prepared using 0.7 eq of 4-cyanopyridine, 0.5 eq of sodium methoxide and 7.2 vol of methanol and stirred at ambient temperature for 2 hrs.

To the solution, 5.5 eq of acetic acid was added and heated to 40° C. for 30 min. Crude ridinilazole produced by the imidate-DAB condensation reaction described in Section 5.2 (above) was dissolved in 9.8 vol of methanol and 4 eq of sodium methoxide. The ridinilazole solution was added to the imidate solution over 5 hours at 40° C. and stirred for 10 hours. The mixture was cooled to ambient temperature over 1 hour and stirred for 1 hour. The slurry was filtered and washed with methanol (2×4.5 vol).

The wet cake was re-slurried in 12 vol of methanol at ambient temperature for 2 hours. The slurry was filtered and washed with methanol (2×4.5 vol). The wet cake was dried at 40° C. to get a recovery of 86%.

The Table below summarizes the LCMS results and shows that the imidate treatment is effective to significantly reduce impurity E.

| Operation | Imp E |
|---|---|
| Crude Ridinilazole | 1895 ppm |
| After Retreatment | 58 ppm |

Any imidate-related impurity introduced by the imidate purging step may be easily removed by carbon treatment (for example as described in Section 5.5.6, below). For example, the product from the imidate retreatment may be dissolved in MeOH upon treatment with NaOMe, the solution treated with carbon to remove imidate-related impurities, and the Impurity E-purged ridinilazole product precipitated out by adding HOAc.

### 5.5.2 Reprecipitation

The level of these entrained Impurities E and F can be reduced by dissolving the crude ridinilazole (thus freeing the entrained Impurities E and F) and then reprecipitating the ridinilazole. This reprecipitation may be conveniently carried out by forming a salt solution (preferably an alkali metal salt solution, e.g. in methanol), followed by reprecipitation of the ridinilazole (e.g. by neutralization, for example by the addition of acetic acid).

Suitable alkali metal salts include sodium, potassium and lithium salts.

Preferred is the dissolution of the crude ridinilazole with sodium methoxide in methanol, followed by precipitation with acetic acid.

Another preferred method is a DMSO/acetic acid reprecipitation/re-slurry (as described in more detail below).

This reprecipitation step may also be used following the imidate treatment (as described in Section 5.5.1, above).

### Exemplary Reprecipitation Process Using NaOMe/HOAc

A wet cake of crude ridinilazole produced by an imidate-DAB condensation reaction as described in Section 5.2 (above) was analysed as described herein and found to contain Impurity E (at 17576 ppm) and Impurity F (at 901 ppm).

NaOMe/HOAc precipitation (based on 200 g DAB) may be carried out as follows.
1) Charge the wet cake into the reactor.
2) Charge Methanol to the same reactor (21.5 vol).
3) Stabilize the temperature of the slurry to 20-25° C.
4) Charge 30% NaOMe/MeOH (4.0 eq) solution over a period of at least 30 min keeping temperature between 20-30° C.
5) Agitate the mixture for at least 30 min at 20-25° C. or until all the solids dissolved.
6) Charge Water (Critical charge, 0.9 vol) to the reactor and stir for at least 30 min.
7) Charge Glacial acetic acid to adjust pH to 5-7 over a period of at least 2 h keeping temperature between 20-25° C.
8) Agitate the slurry for at least 6 h.
9) Filter slurry.
10) Wash cake with methanol (18.0 vol) and pull dry under vacuum at 40° C. for at least 24 h.

This procedure yielded the anhydrous crystalline Form D of ridinilazole characterized by an XRPD pattern substantially in accordance with FIG. 3. It also reduced the levels of Impurities E and F to 4195 and 303 ppm, respectively.

In the above exemplary reprecipitation process, the crude ridinilazole is treated with 4 eq of sodium methoxide and dissolved in methanol. Since ridinilazole has two acidic protons, only two equivalents of sodium methoxide are, in theory, required. The use of 2 eq NaOMe rather than 4 eq in the above example yielded a better purging efficiency of impurities E and F. The reduction in the levels of Impurities E and F by the reprecipitation step may therefore be increased by using stoichiometric quantities of the salt former (here, sodium methoxide).

In the above exemplary reprecipitation processes, the reduction in the levels of Impurities E and F may be further improved by adding an amount of acetic acid required to adjust the pH to between 6-7 (rather than adding a fixed amount).

### Exemplary Reprecipitation Process Using DMSO/HOAc

A wet cake of crude ridinilazole produced by an imidate-DAB condensation reaction as described in Section 5.2 (above) was analysed as described herein and found to contain Impurity E (at 17576 ppm) and Impurity F (at 901 ppm).

A 5 g sample of this crude ridinilazole composition was slurried in 50 mL DMSO and the pH of the mixture was adjusted from 11.7 to 6.9 using 25.43 g of HOAc. The mixture was heated to 100° C. and cooled to ambient.

This procedure yielded the anhydrous crystalline Form D of ridinilazole characterized by an XRPD pattern substantially in accordance with FIG. 3. It also reduced the levels of Impurities E and F to 248 and 81 ppm, respectively.

### 5.5.3 Recrystallization

The differential solubility of ridinilazole and Impurities E and F can be exploited in recrystallization procedures in which ridinilazole is crystalized from a solution containing dissolved Impurities E and F, thereby permitting the separation of ridinilazole from the dissolved impurities.

Thus, the removing step (b) may comprise the step of dissolving the crude ridinilazole composition in a high boiling aprotic solvent and then recrystallizing the ridinilazole.

In preferred embodiments, the high boiling aprotic solvent is DMSO.

In other preferred embodiments, the removing step (b) further comprises slow cooling and/or temperature cycling of the solution.

The invention therefore contemplates the use of a ridinilazole recrystallization step for reducing the levels of Impurities E and/or F in which a composition comprising a mixture of ridinilazole and Impurities E and F is heated in DMSO such that the ridinilazole enters, and subsequently comes out of, solution.

The purging effect of the this process on Impurities E and F may be improved by slow cooling and temperature cycling, and those skilled in the art will be readily able to optimize these parameters by reference to the starting material (see below) and the levels of Impurities E and F present in the ridinilazole mixture.

Such a recrystallization step may be used following the imidate treatment (as described in Section 5.5.1, above).

Alternatively (or in addition), it may be used after a reprecipitation step (as described in Section 5.5.2, above). For example, it may be used after the steps of imidate treatment followed by reprecipitation (see Section 5.5.1 and 5.5.2, above).

### Exemplary Recrystallization Process

A crude ridinilazole product of an imidate-DAB condensation reaction described in Section 5.2 (above) was analysed and found to contain Impurity E (at 474 ppm) and Impurity F (at 65 ppm). The dry cake (225 g) was charged into a reactor and 20 volumes of DMSO (4950 g) and water (112.5 g, 0.5 vol) were added. The mixture was heated to 100° C. with agitation.

The resultant solution was then cooled to 25° C. over a 2 hour period and stirred for at least 2 hours. The resultant slurry was filtered and the cake washed with DMSO (990 g, 4 vol) and MTBE (2×666 g, 2×4 vol). The solids were pulled dry under vacuum at 40° C. for at least 24 h.

Analysis of the recovered solids revealed that the recrystallization process reduced the levels of Impurities E and F to 5 ppm and 20 ppm, respectively.

In a further experiment, the procedure described above was applied to a composition produced according to Example 12 (below) comprising a mixture of hydrated ridinilazole Form A and Impurity E (36 ppm) and Impurity F (at 318 ppm). Analysis of the recovered solids revealed that the recrystallization process reduced the levels of Impurities E and F to 3 ppm and 159 ppm, respectively.

In a yet further experiment, the procedure described above was applied to a composition produced according to Example 12 (below) comprising a mixture of hydrated ridinilazole Form A spiked with Impurity E (to 2036 ppm) and containing Impurity F (at 318 ppm).

Analysis of the recovered solids revealed that the recrystallization process reduced the levels of Impurities E and F to 111 ppm and 124 ppm, respectively.

Impurity purging can be improved by slow cooling and temperature cycling. A composition produced according to Example 12 (below) comprising a mixture of hydrated ridinilazole Form A spiked with Impurity E (to 2036 ppm) and containing Impurity F (at 318 ppm) was used as the starting material.

In a slow cooling experiment, a mixture of this ridinilazole composition and DMSO was heated to 100° C. and held for 4 hours before cooling to ambient over 8 hours. It was found that Impurities E and F were reduced to 306 and 89 ppm, respectively.

In a temperature cycling experiment, the same mixture was heated to 100° C. and held for 1 hour before cooling to ambient over 3 hours and holding for 1 hour. The mixture was then heated to 100° C. over 3 hours and the cooling cycle was repeated three times before holding at ambient for 7 hours. It was found that Impurities E and F were reduced to 117 and 124 ppm, respectively.

5.5.4 Solvent Exchange and/or Crystallisation with Ridinilazole Alkali Metal Salts

The differential solubility of alkali metal salts of ridinilazole (such as the sodium, lithium or potassium salts) in various solvents can be exploited to remove trapped Impurities E and F. For example, the differential solubility of ridinilazole sodium salt in various solvents can be exploited to remove trapped Impurities E and F.

The invention therefore contemplates the use of a ridinilazole sodium salt solvent exchange step for reducing the levels of Impurities E and/or F in which a composition comprising a solution of ridinilazole sodium salt in admixture with Impurities E and F in a first solvent (for example, MeOH) is swapped with a second solvent in which the ridinilazole sodium salt has lower solubility (for example, isopropyl alcohol (IPA)).

For example, upon dissolution of crude ridinilazole with sodium methoxide in methanol, any trapped Impurities E and F are released into solution. Solvent exchange to IPA gradually pushes ridinilazole sodium salt out of solution whilst retaining the impurities in the mother liquor.

When the above process was applied to a composition produced according to Example 12 (below) comprising a mixture of hydrated ridinilazole Form A and Impurities E and F, analysis of the recovered solids revealed that the solvent exchange process reduced the levels of Impurities E and F by 46% and 59%, respectively.

The sodium salt is also quite soluble in DMSO and not as soluble in MTBE. Thus, a crystallisation approach can therefore be applied whereby the sodium salt is dissolved in a suitable solvent (for example methanol or DMSO) and then induced to crystallise by the addition of the solvent in which the salt is less soluble (for example, MTBE)

The purified ridinilazole salt can then be dissolved and anhydrous ridinilazole precipitated (e.g. by addition of acetic acid, as described in Section 5.5.2, above) to yield a purified anhydrous crystalline Form D of ridinilazole characterized by an XRPD pattern substantially in accordance with FIG. 3.

5.5.5 Solvent Exchange with Ridinilazole Lithium Salt

The differential solubility of ridinilazole lithium salt in various solvents can also be exploited to remove trapped Impurities E and F.

The invention therefore contemplates the use of a ridinilazole lithium salt solvent exchange step for reducing the levels of Impurities E and/or F in which a composition comprising a solution of ridinilazole lithium salt in admixture with Impurities E and F in a first solvent is swapped with a second solvent in which the ridinilazole lithium salt has lower solubility.

Ridinilazole lithium salt can be prepared from crude ridinilazole Form D and LiOH in THF/DMSO at 20° C. using a stoichiometry of 1:2 ridinilazole:base. The diffractogram is shown in FIG. 19 and is indicative of a crystalline material. The elevated baseline of the diffractogram may be indicative of some amorphous content and/or it may comprise a DMSO solvate.

### 5.5.6 Carbon Treatment

Impurities E and F may be removed from the crude ridinilazole composition by carbon treatment. Carbon treatment is preferably applied to a solution of the crude ridinilazole mixture, and may comprise contact of such a solution with activated carbon. Suitable solutions include alkali metal ridinilazole salt solutions, for example sodium, potassium or lithium ridinilazole salt solutions.

Treatment with activated carbon preferably further comprises the step of removing said activated carbon by filtration. Alternatively, or in addition, the carbon treatment may comprise recirculation of the solution through an activated carbon filter cartridge.

In preferred embodiments, carbon treatment is preceded by forming an alkali metal salt solution (e.g. in methanol). After carbon treatment of this solution, ridinilazole may be precipitated (e.g. by addition of acetic acid, as described in Section 5.5.2, above). Suitable alkali metal salts include sodium, potassium and lithium salts. Preferred is the dissolution of the crude ridinilazole with sodium methoxide in methanol, followed by carbon treatment and then precipitation with acetic acid.

Any suitable solution and form of activated carbon may be used, including stirring with Norit® SX Plus and recirculation of the solution through an activated carbon filter cartridge (for example, a Zetacarbon R53SP™ cartridge). In the latter case, a carbon loading corresponding to 0.086 Wt may be used with recirculation through the filter for at least 2.5 hours.

Carbon treatment cycles may be repeated while monitoring the levels of Impurities E and F, and continued until the levels are reduced to target levels.

The purified ridinilazole can then be precipitated (e.g. by addition of acetic acid, as described in Section 5.5.2, above) to yield a purified anhydrous crystalline Form D of ridinilazole characterized by an XRPD pattern substantially in accordance with FIG. 3.

### Exemplary Processes Involving Carbon Treatment

According to a first example there is provided a process for producing ridinilazole, or a pharmaceutically acceptable derivative, salt, hydrate, solvate, complex, bioisostere, metabolite or prodrug thereof, the process comprising the steps of:
(a) subjecting 3,3′-diaminobenzidine (DAB) to a condensation reaction to yield said ridinilazole and an intermediate co-product of formula (II):

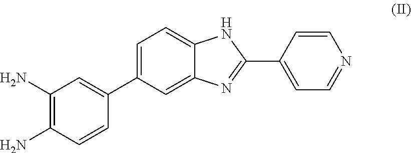 [SMILES: Nc1ccc(-c2ccc3[nH]c(-c4ccncc4)nc3c2)cc1N]

and then
(b) dissolving said ridinilazole and then removing, or reducing the level of, residual DAB and/or the intermediate of formula (II) by treatment of the ridinilazole solution with activated carbon to yield ridinilazole in purified form.

In some embodiments of this example, the DAB of step (a) contains a contaminating aminobenzidine compound (MAB) of formula:

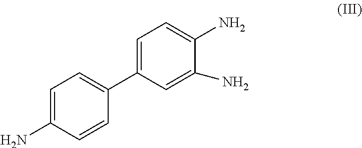 [SMILES: Nc1ccc(-c2ccc(N)c(N)c2)cc1]

The contaminating MAB may be present at ˜0.5% or more, and when subjected to the condensation reaction of step (a) gives rise to an intermediate co-product of formula (IV):

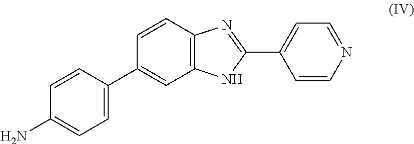 [SMILES: Nc1ccc(-c2ccc3nc(-c4ccncc4)[nH]c3c2)cc1]

Thus, in embodiments where the DAB of step (a) is present in the condensation reaction together with an aminobenzidine contaminant of formula (III):

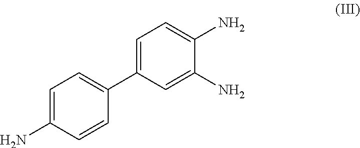 [SMILES: Nc1ccc(-c2ccc(N)c(N)c2)cc1]

so that the condensation reaction yields a further co-product of formula (IV):

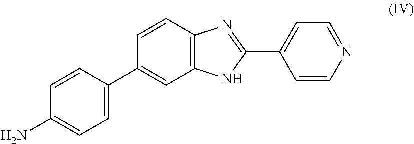 [SMILES: Nc1ccc(-c2ccc3nc(-c4ccncc4)[nH]c3c2)cc1]

the process preferably further comprises removing, or reducing the level of, the compounds of formulae (III) and/or (IV).

The treatment with activated carbon in step (b) may comprise the steps of forming a salt solution of ridinilazole and then treating said solution with activated carbon. Suitable salts include sodium, potassium and lithium salts. Preferred is the sodium salt.

There is broad scope for manipulation of the precise conditions of the imidate-DAB condensation reaction and all such manipulations are within the scope of the invention (as described in Section 5.2, above).

The condensation reaction may be carried out at a temperature of from 10° C. to 100° C. Generally the reaction may be carried out at the reflux temperature of the solvent at normal pressure.

The reaction may be carried out in any suitable solvent that does not interfere with the reaction. Suitable solvents include methanol.

The condensation may comprise:
(a) adding sodium methoxide to 4-cyanopyridine in methanol to produce the imidate compound of formula (V); and then
(b) adding the compound of formula (V) of step (a) to a mixture of DAB and acetic acid in methanol.

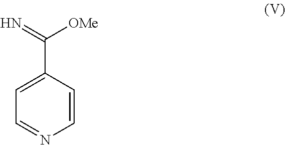

In step (a), other imidates may be produced and used in the condensation reaction by using different alkoxide/alcohol combinations. For example, sodium ethoxide/ethanol may be used instead of sodium methoxide/methanol, while other cations (preferably alkali metals) may replace sodium.

In step (b), other acids, such as TFA, may be used instead of acetic acid.

The present inventors have surprisingly found that despite the use of the highly toxic 3,3′-diaminobenzidine (DAB) and potentially toxic intermediate co-product of formula (II), large scale GMP synthesis of 2,2′-di(pyridin-4-yl)-1H,1′H-5,5′-bibenzo[d]imidazole suitable for use in the formulation of pharmaceutical compositions can be achieved by the use of activated carbon to reduce the aforementioned toxic compounds to acceptable levels

Other exemplary embodiments are as defined in the numbered paragraphs below:

1. A process for producing ridinilazole, or a pharmaceutically acceptable derivative, salt, hydrate, solvate, complex, bioisostere, metabolite or prodrug thereof, the process comprising the steps of:

(a) subjecting 3,3′-diaminobenzidine (DAB) to a condensation reaction to yield said ridinilazole and an intermediate co-product of formula (II):

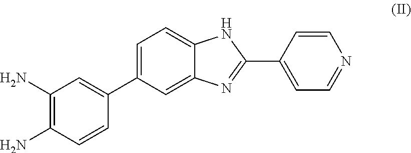 [SMILES: Nc1ccc(-c2ccc3[nH]c(-c4ccncc4)nc3c2)cc1N]

and then
(b) dissolving said ridinilazole and then removing, or reducing the level of, residual DAB and/or the intermediate of formula (II) by treatment of the ridinilazole solution with activated carbon to yield ridinilazole in purified form.

2. The process of paragraph 1 wherein the DAB of step (a) is present in the condensation reaction together with an aminobenzidine contaminant of formula (III):

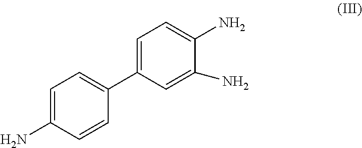 [SMILES: Nc1ccc(-c2ccc(N)c(N)c2)cc1]

so that the condensation reaction yields a further co-product of formula (IV):

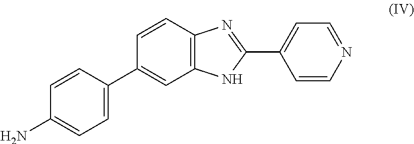 [SMILES: Nc1ccc(-c2ccc3nc(-c4ccncc4)[nH]c3c2)cc1]

and wherein the process further comprises removing, or reducing the level of, the compounds of formulae (III) and/or (IV).

3. The process of paragraph 1 or paragraph 2 wherein the treatment with activated carbon in step (b) further comprises the step of forming a salt, for example a sodium, potassium or lithium salt, solution of ridinilazole and then treating said solution with activated carbon.

4. The process of paragraph 3 wherein the salt solution of ridinilazole is the sodium salt dissolved in methanol.

5. The process of paragraph 4 wherein the sodium salt of ridinilazole is formed by treatment with sodium methoxide.

6. The process of any one of the preceding paragraphs wherein the treatment with activated carbon in step (b) further comprises the step of removing said activated carbon by filtration.

7. The process of any one of paragraphs 1-5 wherein the treatment with activated carbon in step (b) comprises recirculation of the solution through an activated carbon filter cartridge.

8. The process of any one of the preceding paragraphs wherein the treatment with activated carbon in step (b) further comprises the step of acidifying to yield ridinilazole in purified form.

9. The process of any one of the preceding paragraphs wherein the treatment with activated carbon in step (b) comprises the steps of:
(i) forming the sodium salt of ridinilazole in methanol, for example by treatment with sodium methoxide;
(ii) treating the resulting solution of step (i) with activated carbon; and
(iii) acidifying to yield ridinilazole in purified form.

10. The process of any one of the preceding paragraphs wherein the treatment with activated carbon in step (b) reduces the level of intermediate co-product of formula (II) to <100 ppm.

11. The process of any one of paragraphs 2-10 wherein the treatment with activated carbon in step (b) reduces the level of the compound of formula (IV) to <50 ppm.

12. The process of any one of the preceding paragraphs wherein in step (a) said condensation comprises reacting DAB with a compound of formula (V):

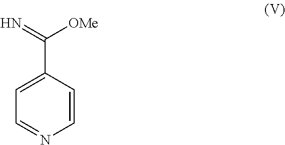

13. The process of paragraph 12 wherein in step (a) said condensation comprises:
(a) adding sodium methoxide to 4-cyanopyridine to produce the compound of formula (V); and then
(b) adding the compound of formula (V) of step (a) to DAB.

14. The process of paragraph 13 wherein in step (a) said condensation comprises:
(a) adding sodium methoxide to 4-cyanopyridine in methanol to produce the compound of formula (V); and then
(b) adding the compound of formula (V) of step (a) to a mixture of DAB and acetic acid in methanol.
(c) adding a mixture of DAB and acetic acid in methanol to the compound of formula (V) of step (a).

15. The process of any one of the preceding paragraphs further comprising the step of isolating ridinilazole by:
(a) mixing the purified ridinilazole in methanol/water to yield a solid;
(b) separating the solid; and
(c) drying the solid.

16. The process of paragraph 15 wherein in step (a) the ratio of methanol:water is 1:2 to 1:4, for example about 1:3.

17. The process of paragraph 15 or paragraph 16 wherein in step (a) the purified ridinilazole is stirred in the methanol/water.

18. The process of any one of paragraphs 15-17 wherein in step (a) the purified ridinilazole is mixed with 10-40, for example about 20, volumes of methanol/water.

19. The process of any one of paragraphs 15-18 wherein in step (b) the solid is separated by filtration.

20. The process of any one of paragraphs 15-19 wherein in step (c) the solid is dried in a filter dryer, optionally wherein the levels of water and/or methanol are monitored.

21. The process of any one of the preceding paragraphs wherein said condensation is carried out at a temperature of 30-80° C.

22. The process of paragraph 21 wherein said condensation is carried out at a temperature of about 60° C.

23. The process of any one of the preceding paragraphs, further comprising the step of forming a pharmaceutically acceptable derivative, salt, hydrate, solvate, complex, bioisostere, metabolite or prodrug of said ridinilazole.

24. The process of paragraph 23 further comprising the step of forming a solvate, for example with DMSO, of said ridinilazole.

25. The process of any one of the preceding paragraphs for producing a pharmaceutical composition, further comprising the step of formulating the purified ridinilazole in a pharmaceutically-acceptable excipient.

26. The process of paragraph 25 further comprising the step of constituting the pharmaceutical formulation into a pharmaceutical kit, pharmaceutical pack or patient pack.

### 5.5.7 Polymorph Conversion

In preferred embodiments, the ridinilazole is present in the crude ridinilazole composition as the anhydrous crystalline Form D characterized by an XRPD pattern substantially in accordance with FIG. 3, and the removing step (b) comprises polymorph conversion from Form D to Form A.

In such embodiments, the polymorph conversion may comprise slurrying the crude ridinilazole composition in an aqueous solvent and then seeding the slurry with crystals of ridinilazole Form A at a water activity (A_{w}) and temperature favouring the crystallization of ridinilazole Form A.

Form A seeds for use in the seeding step may take any physical form. They may therefore be: (a) micronized; (b) in the form of a dry powder; or (c) in the form of a slurry.

The A_{w} is preferably 0.4 and/or the temperature is 2-60° C., more preferably the A_{w} is 0.4-0.5 and the temperature is >2° C. and <30° C., and still more preferably the A_{w} is 0.4-0.5 and the temperature is RT.

Any suitable aqueous solvent may be employed. In preferred embodiments the solvent is MeOH/H₂O.

For example, crude ridinilazole product comprising a mixture of Impurities E and F together with anhydrous crystalline Form D of ridinilazole characterized by an XRPD pattern substantially in accordance with FIG. 3 and prepared according to reaction scheme 1 (above) was converted to ridinilazole polymorph A by slurrying the crude ridinilazole in an aqueous solvent and then seeding the slurry with crystals (which crystals may be micronized, added as a dry powder or in the form of a slurry) of ridinilazole Form A at a water activity (A_{w}) and temperature favouring the crystallization of ridinilazole Form A.

The above exemplary Form D to Form A polymorph conversion procedure was found to reduce the levels of Impurity F by about 60%.

### Exemplary Ridinilazole D to a Polymorph Conversion Process

The conversion can be carried out as follows:
1) Charge Form D.
2) Charge MeOH.
3) Heat to 60° C. Stirred 300 rpm.
4) Hold 15 min.
5) Charge Water over 30 min, a_{w}˜0.47
6) Cool to 40° C. over 2 h.
7) Seeded with 2 wt % Form A (or with 2 wt % Form A in a slurry prepared in MeOH/H₂O (80/20 v/v) and slurried for 2.5 h before addition)
8) Wait 1 h. Thick slurry, limited mobility.
9) Cool to 20° C. over 2 h.
10) Heat to 40° C. over 4 h.
11) Cool to 20° C. over 10 h.
12) Wait 2.5 h. Thick, mobile slurry.
13) VF. Filtration time: 15 sec.
14) Wash reactor 3× with 1 vol MeOH/H₂O (80/20 v/v), 3 ml each wash. Wash wet cake with 1 vol MeOH/H₂O (80/20 v/v), 3 ml.

In the event that the polymorph conversion does not go to completion, or where Form N is produced (perhaps due to local variations in water activity) then a re-slurry process can be carried out. Thus, the polymorph conversion process is preferably preceded by a hot methanol re-slurry step which converts all forms present (including Form N, if present) to Form D.

A preferred methanol re-slurry process is set out below:
Charge Methanol (7.4 vol) in to the reactor.
Charge crude ridinilazole as a wet cake to the reactor.
Heat the slurry to 55-60° C. for at least 3 h.
Cool to 20-25° C. and stir for at least 3 h.
Filter the slurry.
Wash the cake with methanol (2.8 vol) and pull dry under vacuum.
Dry under vacuum at 40° C. for at least 24 h.

### 5.6 Crystalline Forms of Ridinilazole

As explained above, the present inventors have discovered three distinct crystalline forms (polymorphs) of ridinilazole which have particular utility in the above processes and which therefore find application in the efficient large-scale synthesis of ridinilazole for medicinal use (as well as in medicine more generally).

Described herein is a crystalline form of ridinilazole tetrahydrate (Form A) characterized by a powder X-ray diffractogram comprising characteristic peaks at 2-Theta angles of (11.02±0.2)°, (16.53±0.2)° and (13.0±0.2)°.

Also described herein is a crystalline form of ridinilazole tetrahydrate (Form N) characterized by a powder X-ray diffractogram comprising characteristic peaks at 2-Theta angles of (10.82±0.2)°, (13.35±0.2)° and (19.15±0.2)°, optionally comprising characteristic peaks at 2-Theta angles of (10.82±0.2)°, (13.35±0.2)°, (19.15±0.2)°, (8.15±0.2)° and (21.74±0.2)°.

Also described herein is a crystalline form of ridinilazole anhydrate (Form D) characterized by a powder X-ray diffractogram comprising characteristic peaks at 2-Theta angles of (12.7±0.2)°, (23.18±0.2)° and (27.82±0.2)°, optionally comprising characteristic peaks at 2-Theta angles of (12.7±0.2)°, (23.18±0.2)°, (27.82±0.2)°, (19.5±0.2)° and (22.22±0.2)°.

Other embodiments and aspects of this aspect of the invention are as defined in the following numbered paragraphs:

1. A crystalline form of ridinilazole tetrahydrate (Form A) characterized by a powder X-ray diffractogram comprising characteristic peaks at 2-Theta angles of (11.02±0.2)°, (16.53±0.2)° and (13.0±0.2)°.

2. The crystalline Form A of paragraph 1 characterized by an XRPD pattern substantially in accordance with FIG. 1.

3. The crystalline Form A of paragraph 1 or paragraph 2 which is substantially pure.

4. A composition comprising at least 80%, 90%, 95% or 99% w/w of the crystalline Form A of any one of paragraphs 1-3.

5. A crystalline form of ridinilazole tetrahydrate (Form N) characterized by a powder X-ray diffractogram comprising characteristic peaks at 2-Theta angles of (10.82±0.2)°, (13.35±0.2)° and (19.15±0.2)°, optionally comprising characteristic peaks at 2-Theta angles of (10.82±0.2)°, (13.35±0.2)°, (19.15±0.2)°, (8.15±0.2)° and (21.74±0.2)°.

6. The crystalline Form N of paragraph 5 characterized by an XRPD pattern substantially in accordance with FIG. 2.

7. The crystalline Form N of paragraph 5 or paragraph 6 which is substantially pure.

8. A composition comprising at least 80%, 90%, 95% or 99% w/w of the crystalline Form N of any one of paragraphs 5-7.

9. A crystalline form of ridinilazole anhydrate (Form D) characterized by a powder X-ray diffractogram comprising characteristic peaks at 2-Theta angles of (12.7±0.2)°, (23.18±0.2)° and (27.82±0.2)°, optionally comprising characteristic peaks at 2-Theta angles of (12.7±0.2)°, (23.18±0.2)°, (27.82±0.2)°, (19.5±0.2)° and (22.22±0.2)°.

10. The crystalline Form D of paragraph 9 characterized by an XRPD pattern substantially in accordance with FIG. 3.

11. The crystalline Form D of paragraph 9 or paragraph 10 which is substantially pure.

12. A composition comprising at least 80%, 90%, 95% or 99% w/w of the crystalline Form D of any one of paragraphs 9-11.

13. The crystalline form or composition of any one of the preceding paragraphs wherein the XRPD is measured with Cu-Kalpha radiation having a wavelength of 0.15419 nm.

14. The crystalline form or composition of paragraph 13 wherein the XRPD is measured at room temperature.

15. A process for producing the crystalline form or composition as defined in any one of paragraphs 1˜4 comprising the steps of: (a) providing a slurry of ridinilazole Form D in an aqueous solvent; and (b) seeding the slurry with crystals of ridinilazole Form A or Form N at a water activity (A_{w}) and temperature favouring the crystallization of ridinilazole Form A.

16. The process of paragraph 15 wherein the A_{w} is 0.4 and/or the temperature is 2-60° C., optionally wherein the A_{w} is 0.4-0.5 and the temperature is >2° C. and <30° C., for example wherein the A_{w} is 0.4-0.5 and the temperature is RT.

17. A process for producing the crystalline form or composition as defined in any one of paragraphs 5-8 comprising the steps of: (a) providing a slurry of ridinilazole Form D in an aqueous solvent; and (b) seeding the slurry with crystals of ridinilazole Form A or Form N at a water activity (A_{w}) and temperature favouring the crystallization of ridinilazole Form N.

18. The process of paragraph 17 wherein the A_{w} is 0.5 and/or the temperature is 2-60° C., optionally wherein the A_{w} is >0.5 and the temperature is >2° C. and <60° C., for example wherein the A_{w} is >0.55 and the temperature is RT.

19. The process of any one of paragraphs 15-18 wherein the wherein the solvent is MeOH/H₂O.

20. A crystalline form of ridinilazole tetrahydrate obtainable by, or produced by, the process of any one of paragraphs 15-19.

21. A pharmaceutical composition comprising an effective amount of the crystalline form or composition of any one of paragraphs 1-14 or 20 and a pharmaceutically acceptable excipient.

22. The crystalline form or composition of any one of paragraphs 1-14 or 20 for use in therapy or prophylaxis.

23. The crystalline form or composition of any one of paragraphs 1-14 or 20 or pharmaceutical composition of paragraph 21 for use in the therapy or prophylaxis of CDI or CDAD.

24. Use of the crystalline form or composition of any one of paragraphs 1-14 or 20 in the manufacture of a pharmaceutical composition.

### 5.7 Medical Uses

Clostridioides difficile-associated disease (CDAD) defines a set of symptoms and diseases associated with C. difficile infection (CDI). CDAD includes diarrhoea, bloating, flu-like symptoms, fever, appetite loss, abdominal pain, nausea, dehydration and bowel inflammation (colitis). The most serious manifestation of CDAD is pseudomembraneous colitis (PMC), which is manifested histologically by colitis with mucosal plaques, and clinically by severe diarrhoea, abdominal cramps and systemic toxicity. The ridinilazole polymorphs/crystalline forms and pharmaceutical compositions of the invention find application in the treatment of all forms of CDAD, including diarrhoea, bloating, flu-like symptoms, fever, appetite loss, abdominal pain, nausea, dehydration, colitis and pseudomembraneous colitis.

### 5.8 Posology

The pharmaceutical compositions of the present invention can be administered by oral or parenteral routes, including intravenous, intramuscular, intraperitoneal, subcutaneous, transdermal, airway (aerosol), rectal, vaginal and topical (including buccal and sublingual) administration.

The amount of the pharmaceutical composition administered can vary widely according to the particular dosage unit employed, the period of treatment, the age and sex of the patient treated, and the nature and extent of the disorder treated.

In general, the effective amount of the pharmaceutical composition administered will generally range from about 0.01 mg/kg to 10000 mg/kg daily. A unit dosage may contain from 0.05 to 500 mg of ridinilazole, and can be taken one or more times per day.

The preferred route of administration is oral administration. In general a suitable dose will be in the range of 0.01 to 500 mg per kilogram body weight of the recipient per day.

The desired dose is preferably presented as a single dose for daily administration. However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

In determining an effective amount or dose, a number of factors are considered by the attending physician, including, but not limited to, the potency and duration of action of the compounds used, the nature and severity of the illness to be treated, as well as the sex, age, weight, general health and individual responsiveness of the patient to be treated, and other relevant circumstances. Those skilled in the art will appreciate that dosages can also be determined with guidance from Goodman & Goldman's The Pharmacological Basis of Therapeutics, Ninth Edition (1996), Appendix II, pp. 1707-1711.

The effectiveness of a particular dosage of the pharmaceutical composition of the invention can be determined by monitoring the effect of a given dosage on the progression of the CDI and/or CDAD.

### 5.9 Formulation

Pharmaceutical compositions can include stabilizers, antioxidants, colorants and diluents. Pharmaceutically acceptable carriers and additives are chosen such that side effects from the pharmaceutical compound are minimized and the performance of the compound is not compromised to such an extent that treatment is ineffective.

Oral (intra-gastric) is a typical route of administration. Pharmaceutically acceptable carriers can be in solid dosage forms, including tablets, capsules, pills and granules, which can be prepared with coatings and shells, such as enteric coatings and others well known in the art. Liquid dosage forms for oral administration include pharmaceutically acceptable emulsions, solutions, suspensions, syrups and elixirs.

When administered, the pharmaceutical composition can be at or near body temperature.

Compositions intended for oral use can be prepared according to any method known in the art for the manufacture of pharmaceutical compositions and such compositions can contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. Tablets can be uncoated or they can be coated by known techniques, for example to delay disintegration and absorption in the gastrointestinal tract and thereby provide sustained action over a longer period. For example, a time delay material such as glyceryl monostearate or glyceryl distearate can be employed.

Formulations for oral use can also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin or olive oil.

Aqueous suspensions can be produced that contain the active materials in a mixture with excipients suitable for the manufacture of aqueous suspensions. Such excipients include suspending agents, for example, sodium carboxymethylcellulose, methylcellulose, hydroxypropylmethylcellulose, sodium alginate, polyvinylpyrrolidone, gum tragacanth and gum acacia; dispersing or wetting agents can be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

Aqueous suspensions can also contain one or more preservatives, for example, ethyl or n-propyl p-hydroxybenzoate, one or more colouring agents, one or more flavouring—agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol.

Sweetening agents, such as those set forth above, and flavouring agents can be added to provide a palatable oral preparation. These compositions can be preserved by addition of an antioxidant such as ascorbic acid.

Dispersible powders and granules suitable for preparation of an aqueous suspension by addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, can also be present.

Syrups and elixirs containing the ridinilazole can be formulated with sweetening agents, for example glycerol, sorbitol, or sucrose. Such formulations can also contain a demulcent, a preservative and flavouring and colouring agents.

Compositions of the present invention can optionally be supplemented with additional agents such as, for example, viscosity enhancers, preservatives, surfactants and penetration enhancers. Viscosity-building agents include, for example, polyvinyl alcohol, polyvinyl pyrrolidone, methylcellulose, hydroxypropylmethylcellulose, hydroxyethylcellulose, carboxymethylcellulose, hydroxypropylcellulose or other agents known to those skilled in the art. Such agents are typically employed at a level of about 0.01% to about 2% by weight of a pharmaceutical composition.

Preservatives are optionally employed to prevent microbial growth prior to or during use. Suitable preservatives include polyquaternium-1, benzalkonium chloride, thimerosal, chlorobutanol, methylparaben, propylparaben, phenylethyl alcohol, edetate disodium, sorbic acid, or other agents known to those skilled in the art. Typically, such preservatives are employed at a level of about 0.001% to about 1.0% by weight of a pharmaceutical composition.

Solubility of components of the present compositions can be enhanced by a surfactant or other appropriate cosolvent in the composition. Such cosolvents include polysorbates 20, 60 and 80, polyoxyethylene/polyoxypropylene surfactants (e. g., Pluronic F-68, F-84 and P-103), cyclodextrin, or other agents known to those skilled in the art. Typically, such cosolvents are employed at a level of about 0.01% to about 2% by weight of a pharmaceutical composition.

Pharmaceutically acceptable excipients and carriers encompass all the foregoing and the like. The above considerations concerning effective formulations and administration procedures are well known in the art and are described in standard textbooks. See for example Remington: The Science and Practice of Pharmacy, 20th Edition (Lippincott, Williams and Wilkins), 2000; Lieberman et al., ed., Pharmaceutical Dosage Forms, Marcel Decker, New York, N. Y. (1980) and Kibbe et al., ed., Handbook of Pharmaceutical Excipients (3rd Edition), American Pharmaceutical Association, Washington (1999).

Thus, in embodiments where the compound of the invention is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while cornstarch and alginic acid are suitable disintegrating agents. Binding agents may include starch and gelatin, while the lubricating agent, if present, will generally be magnesium stearate, stearic acid or talc. The pharmaceutical compositions may take any suitable form, and include for example tablets, elixirs, capsules, solutions, suspensions, powders, granules, nail lacquers, varnishes and veneers, skin patches and aerosols.

The pharmaceutical composition may take the form of a kit of parts, which kit may comprise the composition of the invention together with instructions for use and/or a plurality of different components in unit dosage form.

For oral administration the pharmaceutical composition of the invention can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch. Tablets for oral use may include pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound of the invention is mixed with a solid diluent, and soft gelatin capsules wherein the active ingredient is mixed with water or an oil such as peanut oil, liquid paraffin or olive oil.

Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

The pharmaceutical compositions of the invention may be tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, colouring agents, and flavouring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

### 6. BRIEF DESCRIPTION OF THE FIGURES

FIG. 1 shows a representative x-ray powder diffraction pattern for ridinilazole tetrahydrate Form A;

FIG. 2 shows a representative x-ray powder diffraction pattern for ridinilazole tetrahydrate Form N;

FIG. 3 shows a representative x-ray powder diffraction pattern for ridinilazole anhydrate Form D;

FIG. 4 shows the phase diagram of ridinilazole in MeOH/H₂O;

FIG. 5 shows the asymmetric unit content of ridinilazole tetrahydrate Form N;

FIG. 6 shows the hydrogen bonding pattern of ridinilazole tetrahydrate Form N;

FIG. 7 shows an ORTEP plot for the ridinilazole and water molecules of the Form A structure;

FIGS. 8-10 show packing diagrams for the ridinilazole Form A structure along each crystallographic axis;

FIG. 11 shows an ORTEP plot for the ridinilazole molecule of the Form D structure;

FIGS. 12-14 show packing diagrams for the ridinilazole Form D structure along each crystallographic axis;

FIG. 15 shows hydrogen bonding between ridinilazole Form D molecules generating a two dimensional network along the ab plane (i.e. as viewed along the c axis);

FIG. 16 shows the conformation of the ridinilazole molecule in Form A (syn), Form N (anti) and Form D (anti);

FIG. 17 shows ridinidazole Form N (top) and A (bottom) both viewed along the a axis to show water channels. Circled are water channels containing 2 independent water molecules, and those containing 4 independent water molecules.

FIG. 18 shows an XRPD overlay of ridinilazole tablet (upper trace), placebo (middle trace) and Form A (lower trace) between ˜10° 2Theta and ˜25° 2Theta.

FIG. 19 shows a representative x-ray powder diffraction pattern for ridinilazole lithium salt.

### 7. EXEMPLIFICATION

The invention will now be described with reference to specific Examples. These are merely exemplary and for illustrative purposes only: they are not intended to be limiting in any way to the scope of the monopoly claimed or to the invention described.

### Methods

### Water Activity (A_{w})

Water activity coefficient and water activity were calculated using UNIFAC Activity Coefficient Calculator (Choy, B.; Reible, D. (1996). UNIFAC Activity Coefficient Calculator (Version 3.0, 1996) [Software]. University of Sydney, Australia and Louisiana State University, USA).

### X-Ray Powder Diffraction (XRPD)

XRPD analyses were performed using a Panalytical Xpert Pro diffractometer equipped with a Cu X-ray tube and a Pixcel detector system. The isothermal samples were analysed in transmission mode and held between low density polyethylene films. The XRPD program used range 3-40° 2θ, step size 0.013°, counting time 99 sec, ˜22 min run time. XRPD patterns were sorted using HighScore Plus 2.2c software.

Carbon (Norit®) treatment: Crude ridinilazole is dissolved in methanol plus 30% sodium methoxide, the resulting solution treated with Norit® SX Plus (0-0.5 wt) and the mixture stirred. The Norit® is then removed by filtration through a filter aid. To the filtrate is then added water followed by acetic acid in order to precipitate purified ridinilazole.

### Example 1: Production of Ridinilazole Form A

Reaction: The reaction flask was charged with 4-cyano-pyridine (0.85 kg), and MeOH (5.4 kg) and NAM-30 (NaOMe as 30 wt % solution in MeOH; 0.5 eq; 0.15 kg) was dosed in. The resulting mixture was heated at 60° C. for 10 min. and then cooled. This solution was added to a mixture of 3,3′-diaminobenzidine (DAB) (0.35 kg) and acetic acid (0.25 kg) in MeOH (1 l) at 60° C. in 1 h. The mixture was then heated for 2 h. The reaction mixture was allowed to cool to ambient temperature overnight. The crystalline mass was filtered and washed with MeOH (1.4 L) and sucked dry on the filter.

Purification: The Norit treatment was conducted 4 times.

Polymorph formation: The reslurry in 20 vols of 1:3 WFI water:MeOH afforded the desired polymorph, drying was conducted in a vacuum drying oven @ ambient temperature and a nitrogen purge for 6 days.

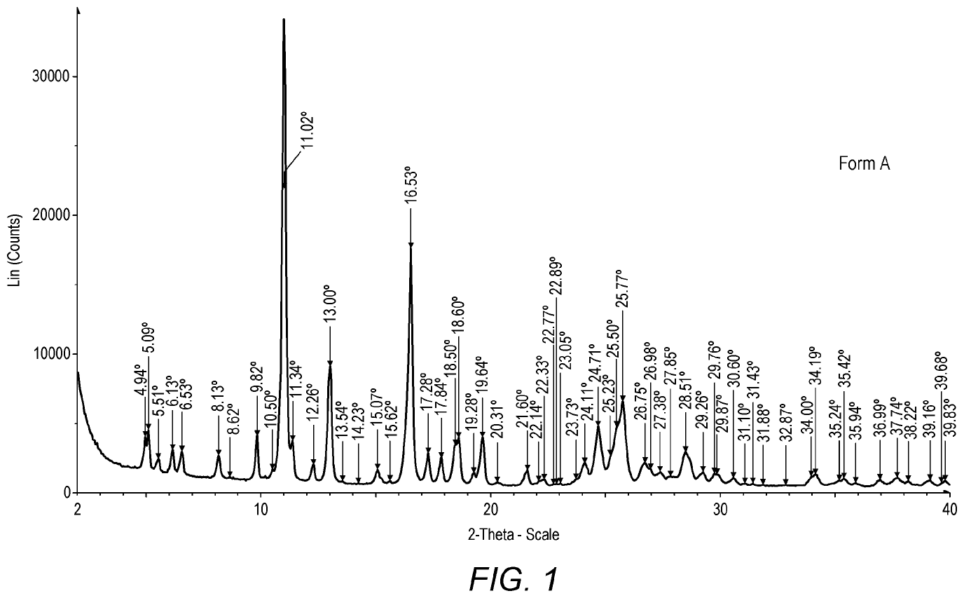

XRPD analysis showed that this process yielded hydrated ridinilazole Form A (see FIG. 1). The reflections are shown in the table below:

Angle 2-Theta°
(Form A)

4.94
5.09
5.51
6.13
6.53
8.13
8.62
9.82
10.5
11.02
11.34
12.26
13
13.54
14.23
15.07
15.62
16.53
17.28
17.84
18.5
18.6
19.28
19.64
20.31
21.6
22.14
22.33
22.77
22.89
23.05
23.73
24.11
24.71
25.23
25.5
25.77
26.75
26.98
27.38
27.85
28.51
29.26
29.76
29.87
30.6
31.1
31.43
31.88
32.87
34
34.19
35.24
35.42
35.94
36.99
37.74
38.22
39.16
39.68
39.83

### Example 2: Production of Ridinilazole Form N

Pattern N material was isolated from a crystallisation development experiment carried out in methylacetate/water (15 vols, 95.3:4.7% v/v). Ridinilazole (5.0 g) was heated to 50° C. in methyl acetate. Water was added and the mixture held at 50° C. for 1 hr before cooling to ambient at 0.2° C./min.

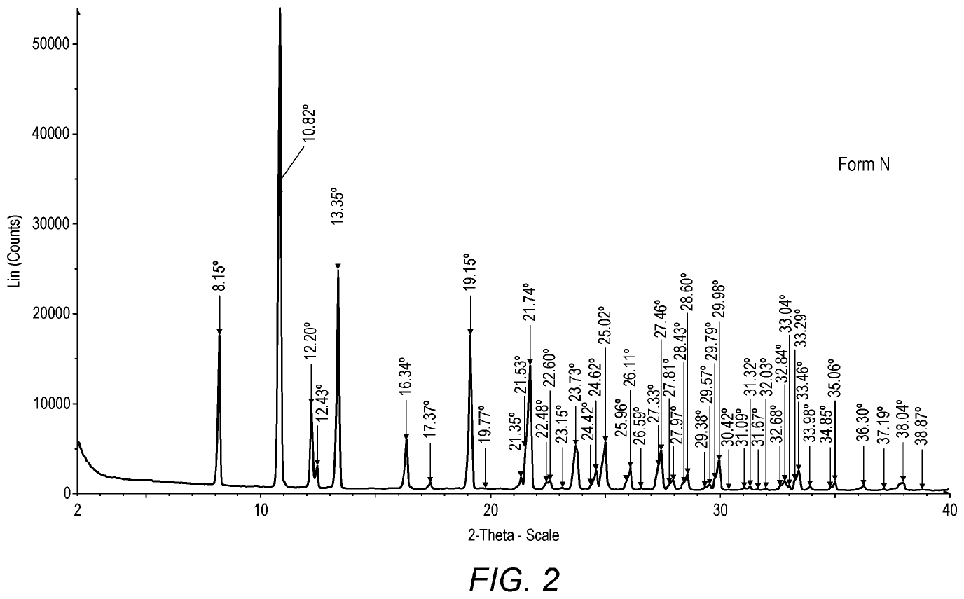

XRPD analysis showed that this process yielded hydrated ridinilazole Form N (see FIG. 2). The reflections are shown in the table below:

Angle 2-Theta°
(Form N)

8.15
10.82
12.2
12.43
13.35
16.34
17.37
19.15
19.77
21.35
21.53
21.74
22.48
22.6
23.15
23.73
24.42
24.62
25.02
25.96
26.11
26.59
27.33
27.46
27.81
27.97
28.43
28.6
29.38
29.57
29.79
29.98
30.42
31.09
31.32
31.67
32.03
32.68
32.84
33.04
33.29
33.46
33.98
34.85
35.06
36.3
37.19
38.04
38.87

### Example 3: Production of Ridinilazole Form D

Reaction: The reaction flask was charged with 4-cyano-pyridine (0.85 kg), and MeOH (5.4 kg) and NaOMe as 30 wt % solution in MeOH; 0.5 eq; 0.15 kg (NAM-30) was dosed in. The resulting mixture was heated at 60° C. for 10 min. and then cooled. This solution was added to a mixture of DAB (0.35 kg) and acetic acid (0.25 kg) in MeOH (1 l) at 60° C. in 1 h. The mixture was then heated for 2 h. The reaction mixture was allowed to cool to ambient temperature overnight. The crystalline mass was filtered and washed with MeOH (1.4 L) and sucked dry on the filter.

Purification: The Norit® treatment was conducted 4 times.

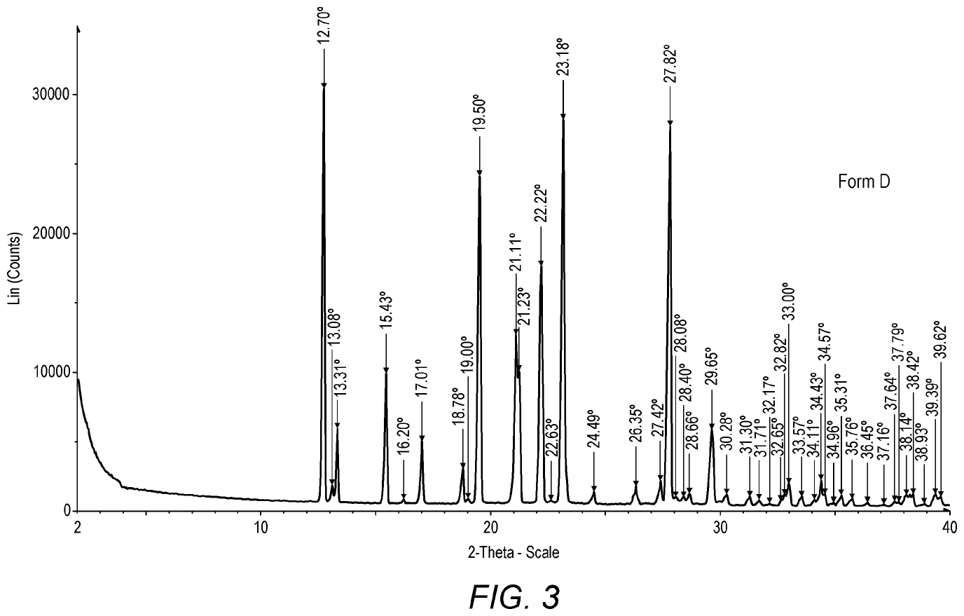

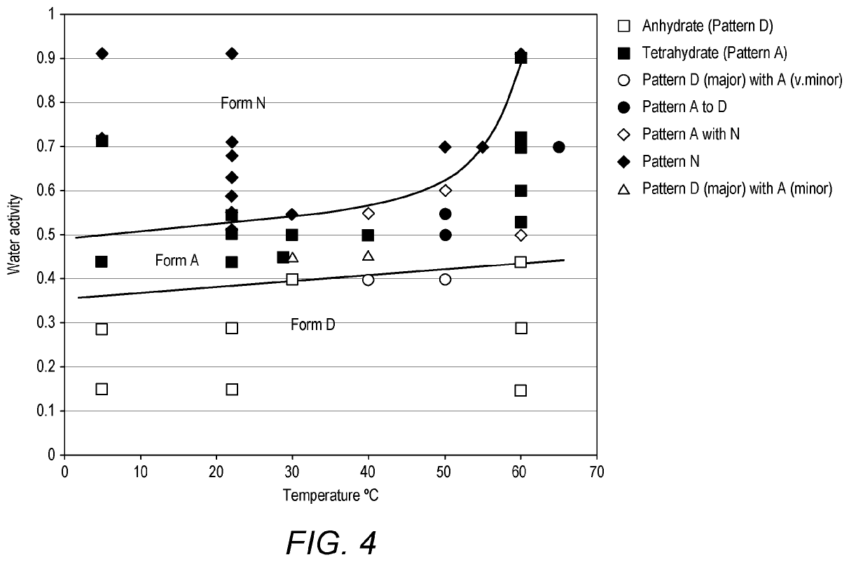

XRPD analysis showed that this process yielded ridinilazole anhydrate Form D (see FIG. 3). The reflections are shown in the table below:

Angle 2-Theta°
(Form D)

12.7
13.08
13.31
15.43
16.2
17.01
18.78
19
19.5
21.11
21.23
22.22
22.63
23.18
24.49
26.35
27.42
27.82
28.08
28.4
28.66
29.65
30.28
31.3
31.71
32.17
32.65
32.82
33
33.57
34.11
34.43
34.57
34.96
35.31
35.76
36.45
37.16
37.64
37.79
38.14
38.42
38.93
39.39
39.62

### Example 4: Conversion of Ridinilazole Form D to Form A

Ridinilazole Form D is prepared as described in Example 3. Ridinilazole Form A is prepared as described in Example 1. Seed crystals were prepared by hand grinding and sifting. The conversion was carried out as follows:
1) Charge Form D.
2) Charge MeOH.
3) Heat to 60° C. Stirred 300 rpm.
4) Hold 15 min.
5) Charge Water over 30 min, a_{w}-0.47
6) Cool to 40° C. over 2 h.
7) Seeded with 2 wt % Form A (or with 2 wt % Form A in a slurry prepared in MeOH/H₂O (80/20 v/v) and slurried for 2.5 h before addition)
8) Wait 1 h. Thick slurry, limited mobility.
9) Cool to 20° C. over 2 h.
10) Heat to 40° C. over 4 h.
11) Cool to 20° C. over 10 h.
12) Wait 2.5 h. Thick, mobile slurry.
13) VF. Filtration time: 15 sec.
14) Wash reactor 3× with 1 vol MeOH/H₂O (80/20 v/v), 3 ml each wash. Wash wet cake with 1 vol MeOH/H₂O (80/20 v/v), 3 ml.

### Example 5: Conversion of Ridinilazole Form D to Form N

Ridinilazole Form D is prepared as described in Example 3. Ridinilazole Form A is prepared as described in Example 1. Seed crystals were prepared by hand grinding and sifting. After approximately 20 minutes, microscopy images indicated mostly smaller agglomerates (˜20 μm), although some larger agglomerates were still present (˜80 μm). XRPD analysis indicated the material was still composed of Form A.

The conversion was carried out at a 3 g scale as shown in the Table below:

1) Charge Form D.
2) Charge Water (18 ml) and MeOH (22 ml), a_{w} ~0.72.
3) Heated to 50° C. Stirred 300 rpm.
4) Hold 20 min. Mobile slurry.
5) Seed with 0.5 wt % Form A.
6) Hold l h. Mobile slurry.
7) Charge MeOH 50 ml over 167 min, a_{w} ~0.47.
8) Wait l h. Mobile slurry.
9) Cool to 20° C. over 15 h.
10) VF. Minimal solids left on walls of reactor and impeller.
11) Washed reactor with 1 filter cake vol MeOH/H₂O (80/20 v/v), 13 ml.
Washed wet cake with 1 filter cake vol MeOH/H₂O (80/20 v/v), 13 ml.
12) Air dried. RT, 1 d.
^{a}Masses, volumes, weight percentages, and temperatures are approximate.
^{b}EasyMax Equipment: 100 ml glass reactor. Stainless steel pitch blade impeller, 3.8 cm. Cold water condenser.

The slurry was relatively thin compared to that formed in Example 4, and it remained mobile throughout the entire period. No discoloration (indicating the presence of Form D, which is brown) was observed.

The data above show that ridinilazole Form N exhibits improved rheology under seeded slurry processing conditions which may speed filtration and improve deliquoring at larger scales.

### Example 6: Crystal Structure of Ridinilazole Tetrahydrate Form N

Single crystals of ridinilazole Form N of suitable quality for full structure determination were grown via vapour diffusion at 5° C. from a solution of ridinilazole in dioxane/water (82:18% v/v, Aw˜0.83)/DMSO using MEK as antisolvent and the crystal structure was determined with monoclinic crystal system and P21/C space group.

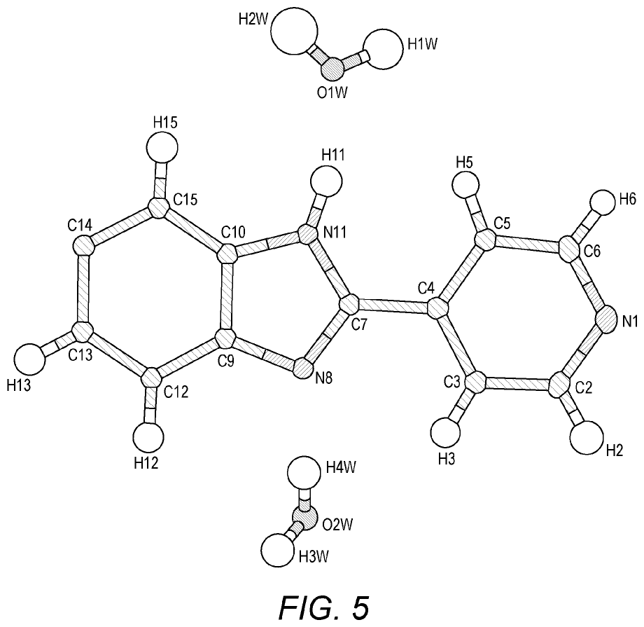

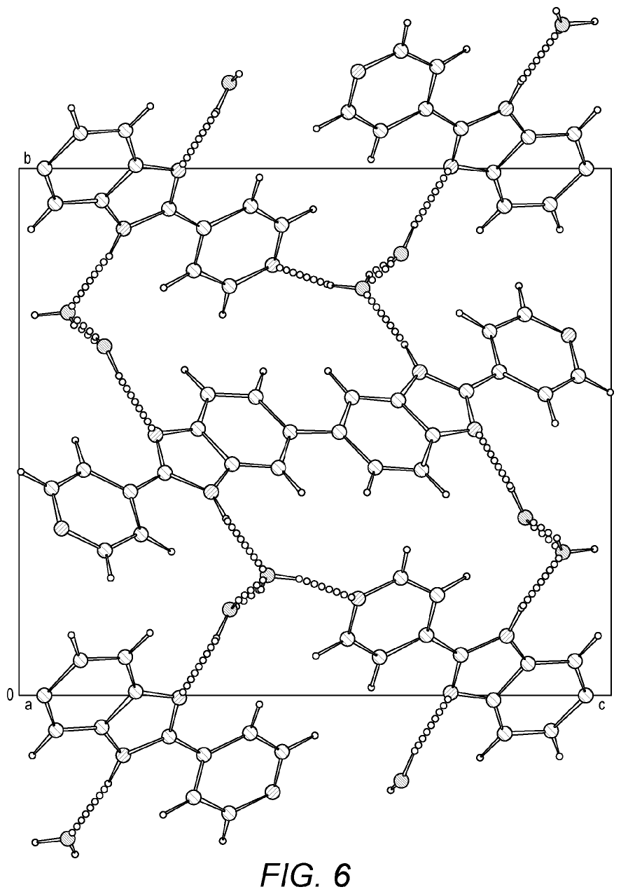

Form N of ridinilazole tetrahydrate was fully solved. The crystal structure is a tetrahydrate which contains half a molecule of ridinilazole and two independent water molecules per asymmetric unit. FIGS. 5 and 6 show respectively the asymmetric unit content and the hydrogen bonding pattern of the determined crystal structure.

### Example 7: Crystal Structure of Ridinilazole Tetrahydrate Form A

Single crystals of ridinilazole Form A were grown via liquid diffusion at RT of a solution of ridinilazole in NMP/dioxane using chloroform as antisolvent. A needle crystal specimen, approximate dimensions 0.380 mm×0.015 mm×0.010 mm, was used for the X-ray crystallographic analysis on beamline 119 at Diamond Light Source.

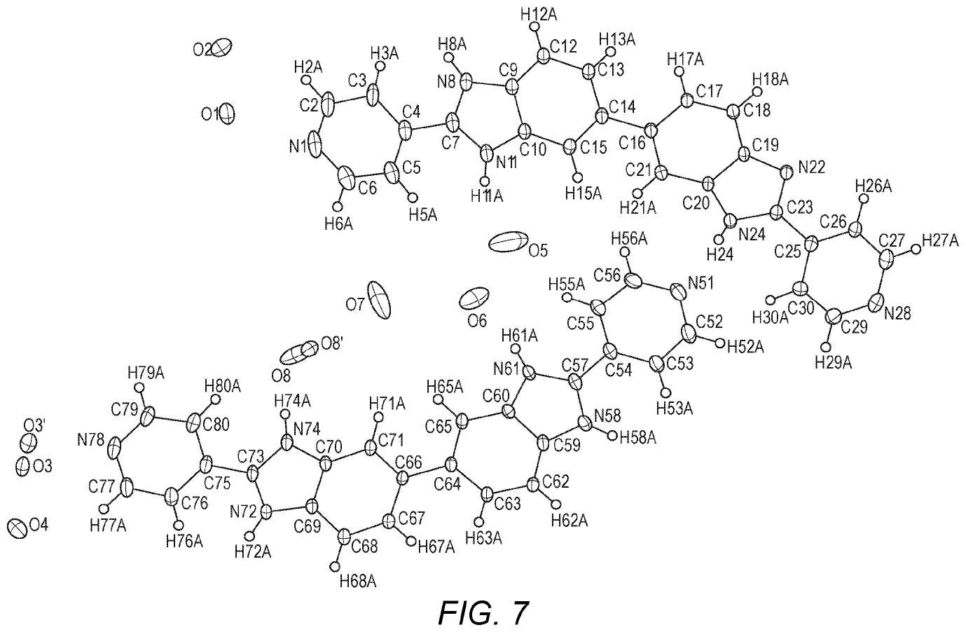

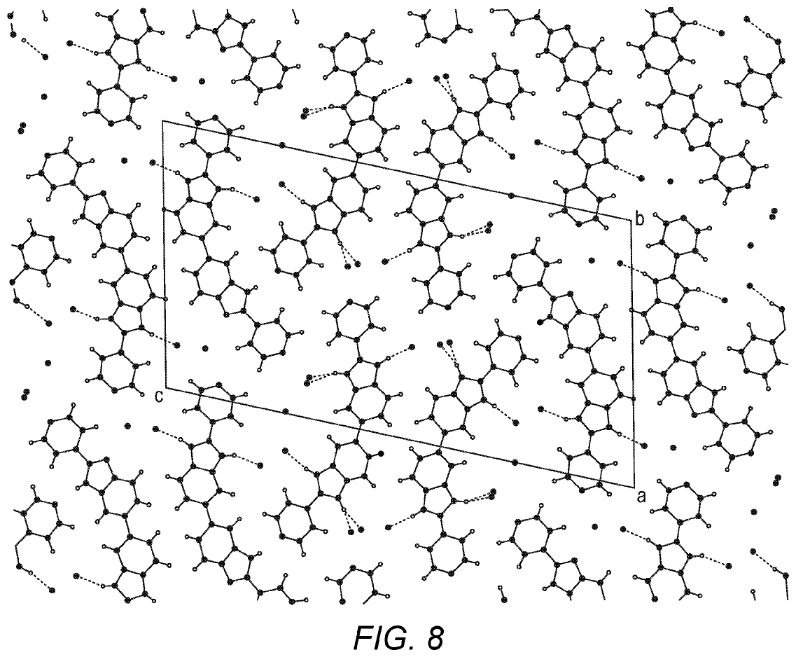

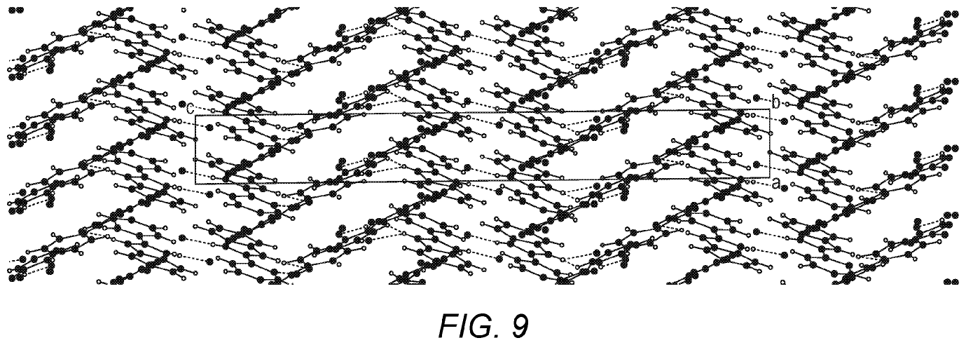

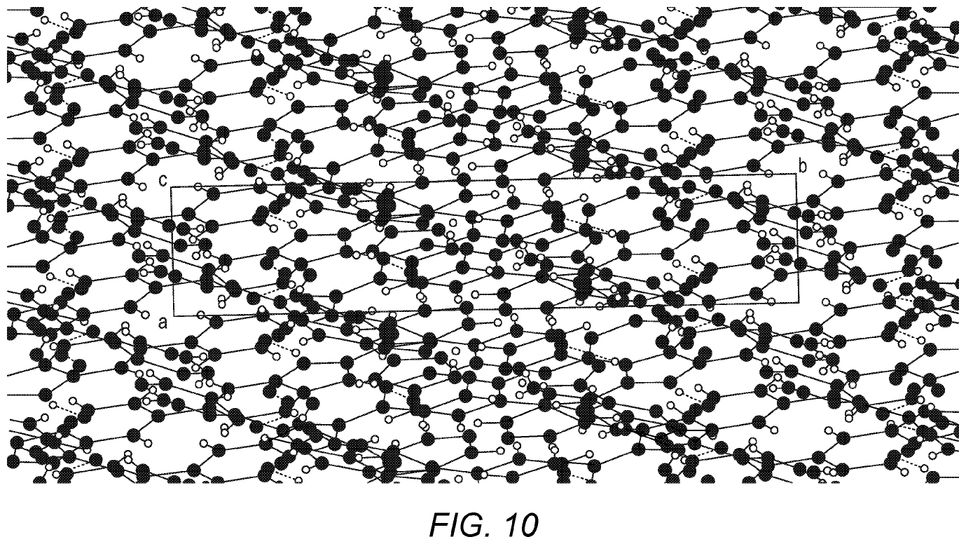

An atom numbering scheme for the ridinilazole and water molecules is displayed in FIG. 7 as an ORTEP plot. Packing diagrams for the ridinilazole Form A structure are displayed in FIG. 8 to FIG. 10 and are shown along each crystallographic axis. Hydrogen bonding between ridinilazole molecules cannot be described as only one hydrogen bond between N24-H24 . . . N51 can be clearly located. The other hydrogen bonds occurring in the structure are formed between the water molecules, imidazole hydrogens and pyridine nitrogen atoms. However due to the large disorder of water molecules and their hydrogen atoms the hydrogen bond network cannot be fully resolved.

### Example 8: Crystal Structure of Ridinilazole Anhydrate Form D

Single crystals of ridinilazole Form D were grown via vapour diffusion at RT of a solution of ridinilazole in ethanol using water as antisolvent and were submitted for single crystal structure determination. A prismatic crystal specimen, approximate dimensions 0.3 mm×0.2 mm×0.1 mm, was used for the X-ray crystallographic analysis.

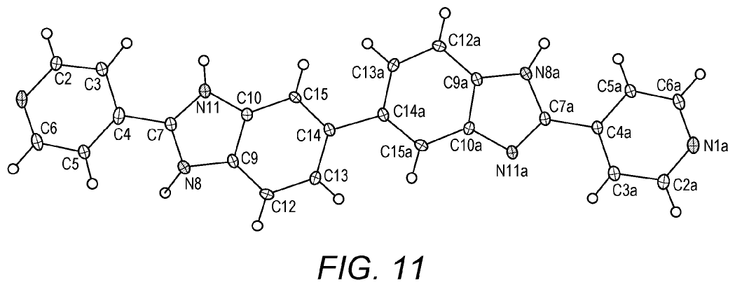

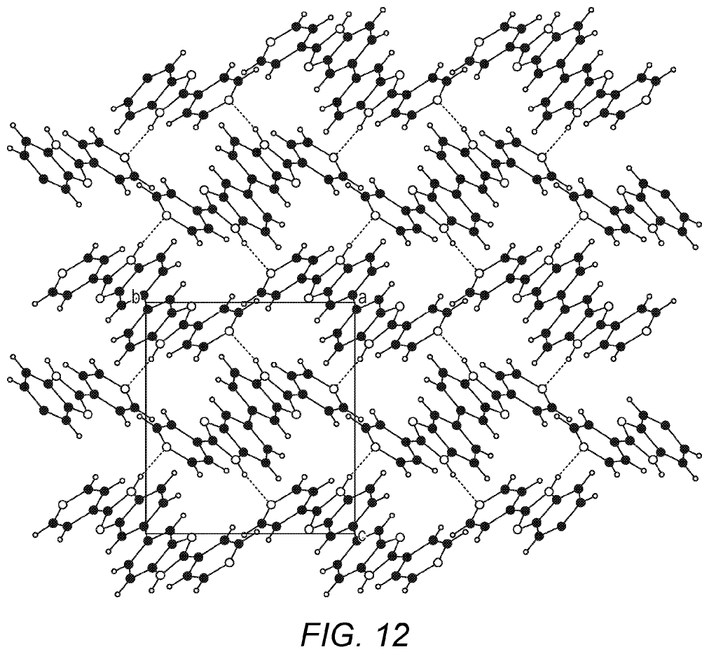

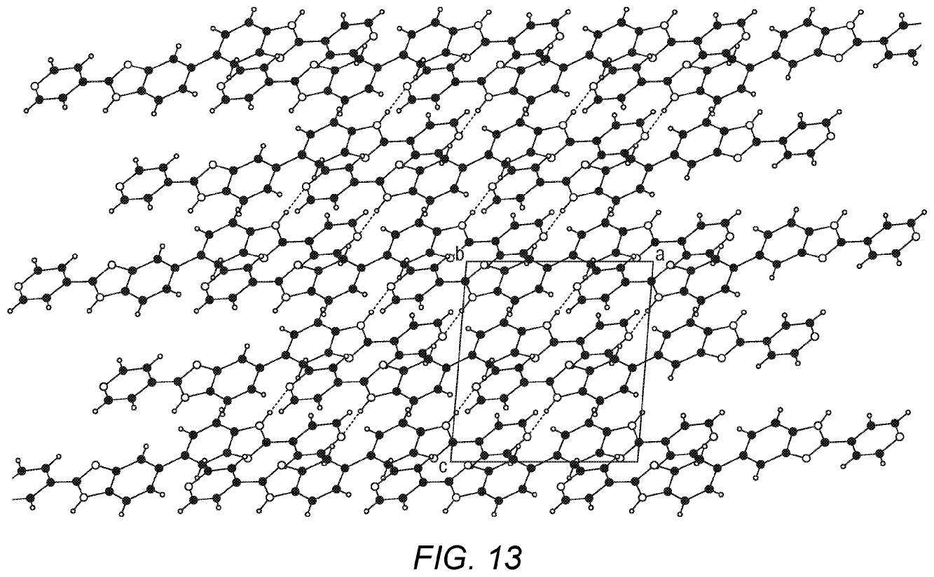

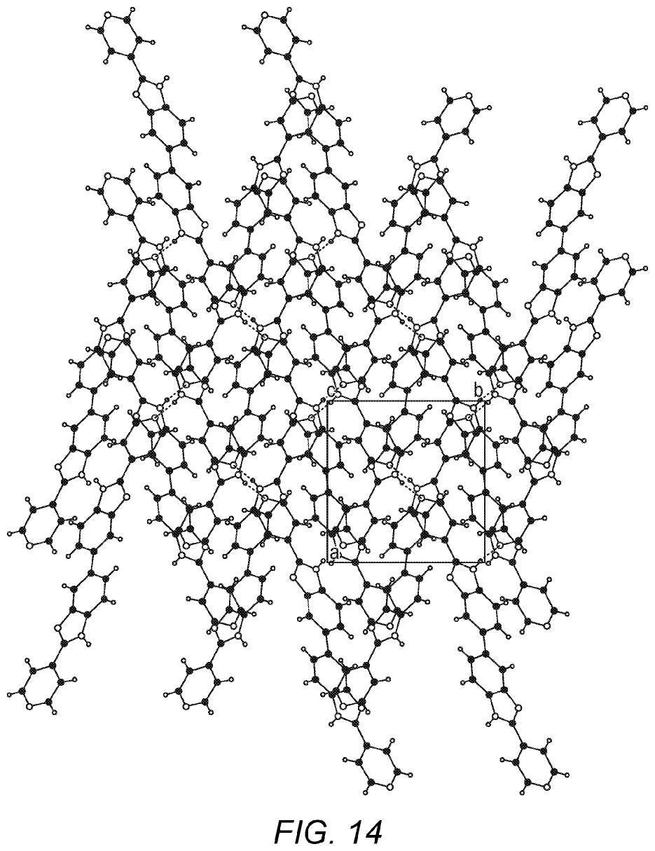

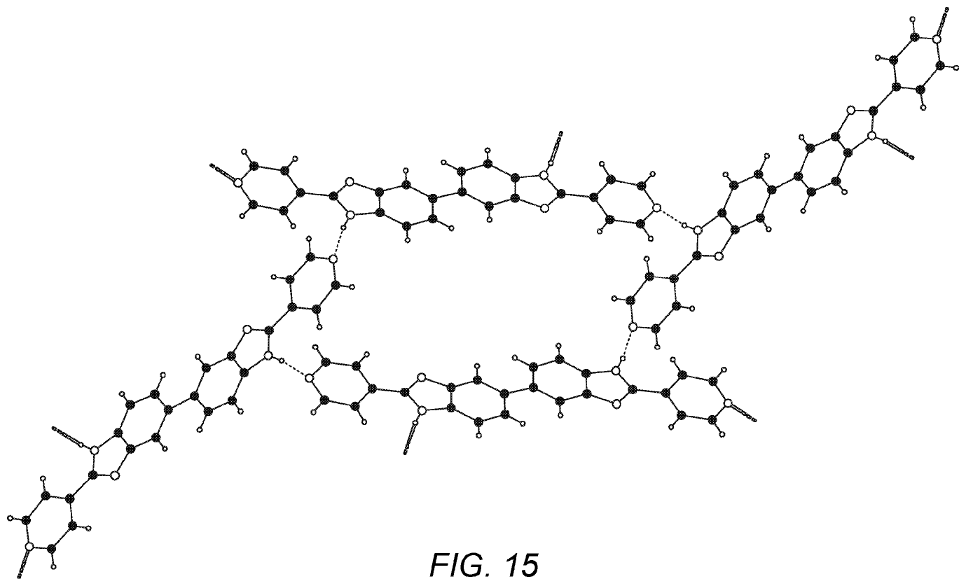

The structure was solved by routine automatic direct methods and refined by least-squares refinement on all unique measured F2 values. The numbering scheme used in the refinement is shown in FIG. 11. An atom numbering scheme for the ridinilazole molecule is displayed in FIG. 11 as an ORTEP plot. Packing diagrams for the ridinilazole Form D structure are displayed in FIGS. 12-14 and are shown along each crystallographic axis. Hydrogen bonding between ridinilazole molecules generate a two dimensional network along the ab plane (see FIG. 15). The hydrogen bonds are formed between the donating hydrogen imidazole nitrogen atoms and the accepting pyridine nitrogen atoms. The network is expanded in the third direction through weaker interaction between hydrogens atoms and π electrons of aromatic carbons.

### Example 9: Comparison of Crystal Structures of Ridinilazole Forms A, N and D

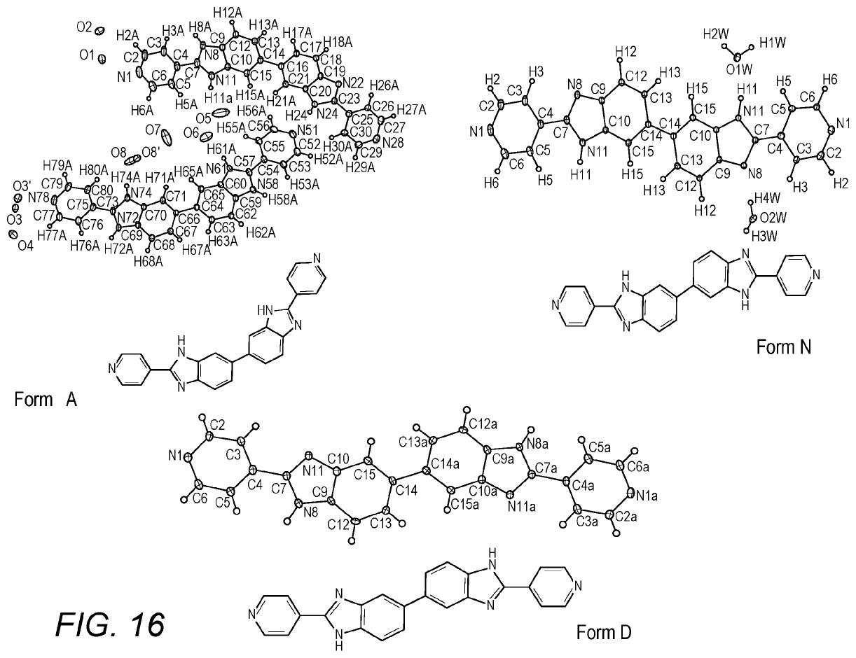

A major difference seen between the three crystal structures is that the conformation of the molecules of ridinilazole are syn in Form A whereas they are anti in Forms N and D (see FIG. 16).

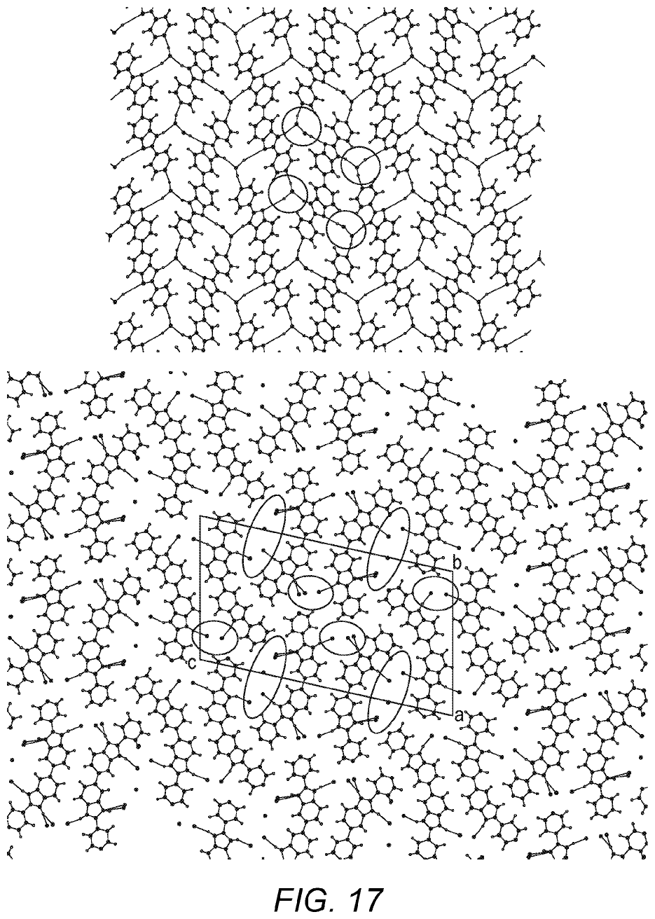

Another difference is in the hydrogen bonding arrangements. Form A shows hydrogen bonding between ridinilazole molecules whereas in Form N ridinidazole molecules interact only with water molecules. In Form A, a larger channel containing four independent water molecules is seen where as in Form N all the channels contain two independent water molecules. In Form D, no water molecules are present and thus the only hydrogen bonds are made between ridinilazole molecules (see FIG. 17).

A major difference is also seen between the torsion angles made in the three structures between the phenyl rings. For Form N and D the torsion angle is equal to 180° and thus the ridinilazole molecule is planar (centre of symmetry between the phenyl rings) whereas for Form A the torsion angles are of 43.0 and 43.3° (two independent molecules). A further major difference between the two structures is that both are different tautomers of ridinilazole, in Form N the hydrogen is bonded to N11 whereas in Form D the hydrogen is bonded to N8 of the imidazole rings. As these are hydrogen bond donating groups in both structures, the packing between both structures is very different.

### Example 10: Ridinilazole Tablet Dosage Form with Form A

Ridinilazole tablets (200 mg) were prepared as described below:

### Wet Granulation

After screening into the high shear granulator bowl, batch quantities of ridinilazole (Form A), lactose monohydrate, microcrystalline cellulose, hydroxypropylcellulose and croscarmellose sodium for the wet granulation, intragranular phase are subject to an initial short premixing of approximately 1 minute at 80 revolutions per minute (rpm).

With continued mixing, purified water is added. At 12% by weight of added water and at 24% by weight of added water the wet mass is transferred manually through a 2000 μm screen to improve water distribution, each time being returned to the granulator bowl to continue granulation. At approximately 35% by weight added water the wet granules are transferred into a fluid bed dryer.

### Drying

The wet granules are dried within the fluid bed dryer at an inlet air temperature of approximately 60° C. until the target limit of detection (LOD) (+0.5% of initial dry blend value) is achieved. Upon completion of the drying. The dried granules are transferred through a Comil equipped with 1143 μm screen into an appropriately sized blender bin.

### Final Blending

When five (or six) dried granulations have been completed they are combined and the calculated batch quantities of lactose monohydrate, microcrystalline cellulose and croscarmellose sodium for the extra granular phase are transferred manually through a 1000 micrometre screen to the 20 L bin containing the dried granules. Blending is performed by tumbling the 20 L bin in the blender for 2 minutes at 30 rpm.

### Lubrication

The calculated batch quantity of magnesium stearate is transferred manually through a 250 micrometre screen into the 20 L bin containing the final blend. Lubrication is performed by tumbling the 20 L bin in the blender for 2 minutes at 30 rpm.

### Compression

Tablets are compressed using oval shaped tooling. Dedusting and metal checking are performed in line post compression.

### Coating

Tablet cores are coated in a pan coater with Opadry® II Brown. Target weight gain for coated tablets is 3 to 4%.

### XRPD Analysis

XRPD analysis was carried out on the ridinilazole tablets to confirm no form change occurred after tableting. One tablet was crushed with a pestle and mortar and analysed by transmission XRPD. Small amounts of the sample coating could not be isolated completely from the crushed sample.

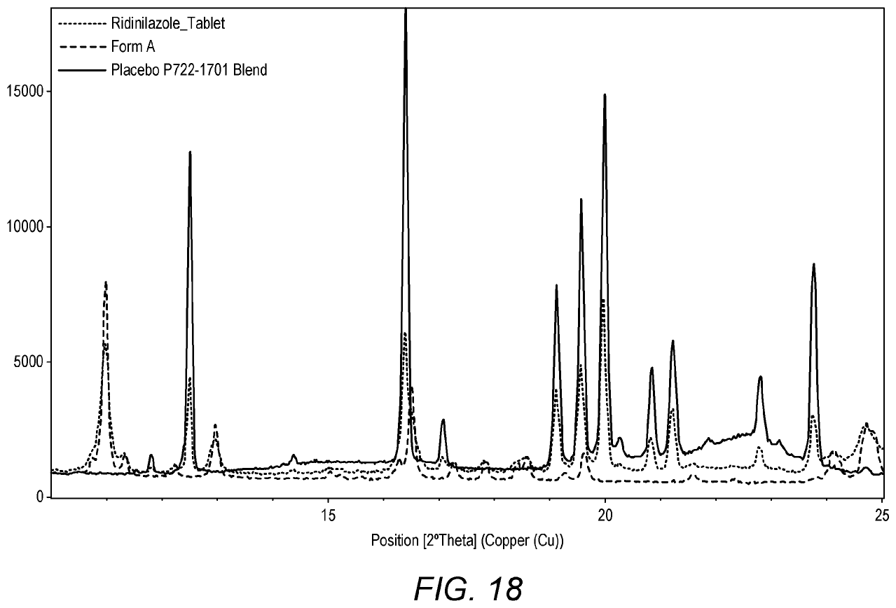

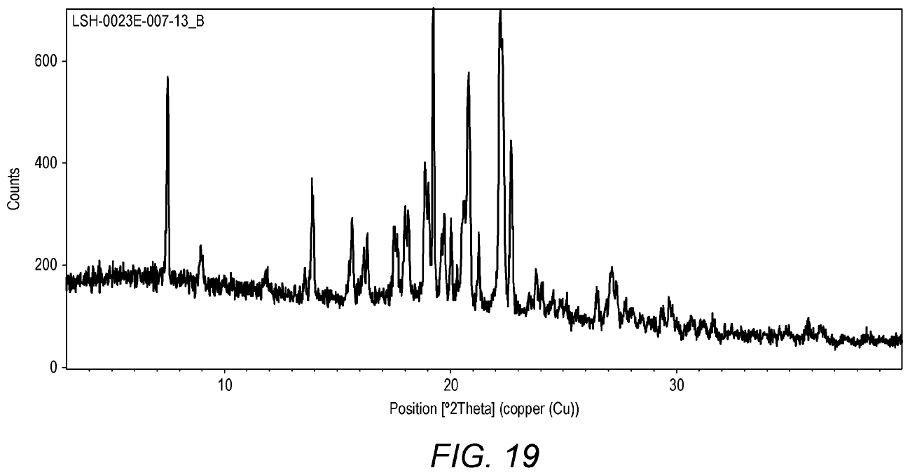

The XRPD trace showed that while the sample compared to Form A with a small amount of peak shifting, there were extra peaks present at ˜12.5° 2Theta and from ˜19-24° 2Theta. XRPD analysis of ridinilazole tablet, ridinilazole Form A and placebo blend confirmed these extra peaks were due to the placebo mixture (FIG. 18) i.e. the extra peaks were present in the placebo mixture (FIG. 18) and so were due to the excipient.

### Example 11: Process for Producing Purified Ridinilazole

Reaction: The reaction flask was charged with 4-cyano-pyridine (0.85 kg), and MeOH (5.4 kg) and NaOMe (as 30 wt % solution in MeOH; 0.5 eq) (0.15 kg) was dosed in. The resulting mixture was heated at 60° C. for 10 min followed by cooling.

The resultant solution, was dosed to a mixture of DAB (0.35 kg) and acetic acid (0.25 kg) in MeOH (1 l) at 60° C. in 1 h, heating for 2 h.

The reaction mixture was allowed to cool to ambient temperature overnight. The mass was then filtered and washed with MeOH (1.4 L) and sucked dry on a filter.

Purification: Crude product, Norit® SX Plus (260 g) and MeOH (6 kg) were charged to a vessel and NaOMe (as 30 wt % solution in MeOH (600 g)) was added. Purification can also be achieved by recirculation of the sodium salt solution through an activated carbon filter cartridge (for example, an R53SP™ cartridge).

The resulting solution was stirred at ambient temperature and was filtered over dicalite and then washed with MeOH (2×500 ml). Water (118 g; 4 eq.) and then acetic acid (206 g) was added to the mixture. The resulting slurry was stirred for two days at ambient temperature. The suspension was filtered, washed with MeOH (1.4 L) and dried o.n. This treatment was conducted 4 times to reduce the intermediate co-product of formula (II):

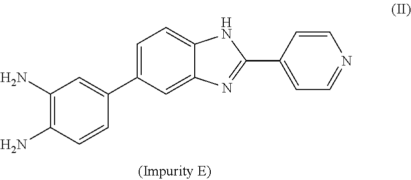 [SMILES: Nc1ccc(-c2ccc3[nH]c(-c4ccncc4)nc3c2)cc1N]

content to a level <100 ppm.

Polymorph formation: A reslurry in 20 vols of 1:3 water:MeOH afforded pure ridinilazole. Drying was conducted in a vacuum drying oven at ambient temperature and a nitrogen purge for 6 days to yield a solid hydrate.

X-ray powder diffraction: X-ray powder diffraction (XRPD) studies were performed on a Bruker AXS D2 PHASER in Bragg-Brentano configuration. Using a Cu anode at 30 kV, 10 mA; sample stage standard rotating; monochromatisation by a Kb-filter (0.5% Ni). Slits: fixed divergence slits 1.0 mm)(=0.61°, primary axial Soller slit 2.5°, secondary axial Soller slit 2.5°. Detector: Linear detector LYNXEYE with receiving slit 5° detector opening. Measurement conditions: scan range 5-45° 2q, sample rotation 5 rpm, 0.5 s/step, 0.010°/step, 3.0 mm detector slit. No background correction or smoothing is applied to the patterns. The contribution of the Cu-Kα2 is stripped off using the Bruker software.

XRPD analysis showed that this process yielded hydrated ridinilazole Form A (see FIG. 1).

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A composition comprising a mixture of compounds, said mixture comprising ridinilazole and compounds of formulae (II) and (IV): 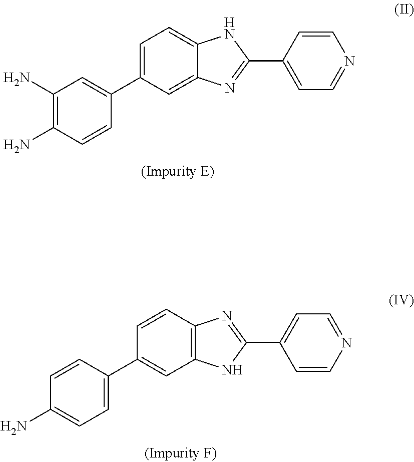 [SMILES: Nc1ccc(-c2ccc3[nH]c(-c4ccncc4)nc3c2)cc1N ; Nc1ccc(-c2ccc3nc(-c4ccncc4)[nH]c3c2)cc1]
wherein the combined amount of Impurities E and F in the mixture is less than 100 ppm.

2. The composition of claim 1, wherein the ridinilazole is a crystalline form of ridinilazole tetrahydrate (Form A) characterized by a powder X-ray diffractogram (XRPD) comprising characteristic peaks at 2-Theta angles of (11.02±0.2)°, (16.53±0.2)° and (13.0±0.2)°.

3. The composition of claim 2 wherein the crystalline Form A is characterized by an XRPD pattern substantially in accordance with FIG. 1.

4. The composition of claim 2 or claim 3 wherein the ridinilazole crystalline Form A is substantially pure.

5. A composition of any one of the preceding claims wherein the mixture comprises at least 80%, 90%, 95% or 99% w/w of the crystalline Form A of any one of claims 2-4.

6. The composition of any one of claims 2-5 wherein the XRPD is measured with Cu-Kalpha radiation having a wavelength of 0.15419 nm.

7. The composition of claim 6 wherein the XRPD is measured at room temperature.

8. The composition of any one of the preceding claims wherein the amount of Impurity E present in the mixture is <50 ppm.

9. The composition of any one of the preceding claims wherein the amount of Impurity F present in the mixture is <50 ppm.

10. The composition of any one of the preceding claims wherein: (a) the amount of Impurity E present in the mixture is <50 ppm; and (b) the amount of Impurity F present in the mixture is <50 ppm.

11. The composition of any one of claims 1-9 wherein the amount of Impurity E or that of Impurity F present in the mixture is greater than 50 ppm but less than 100 ppm.

12. A process for producing a composition as defined in any one of the preceding claims comprising the steps of: (a) providing a crude ridinilazole composition comprising a mixture of compounds, said mixture comprising ridinilazole and compounds of formulae (II) and (IV): 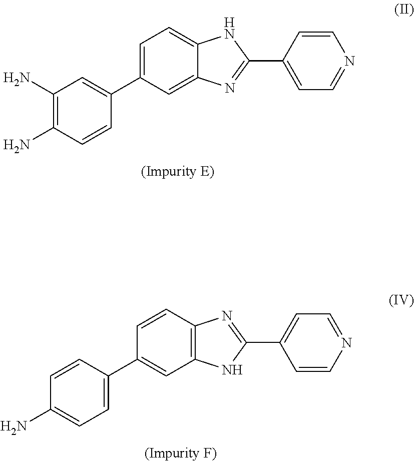 [SMILES: Nc1ccc(-c2ccc3[nH]c(-c4ccncc4)nc3c2)cc1N ; Nc1ccc(-c2ccc3nc(-c4ccncc4)[nH]c3c2)cc1]
wherein the combined amount of the Impurities E and F in the mixture is greater than 100 ppm; and then
(b) removing Impurities E and F from the mixture to produce a purified ridinilazole composition in which the combined amount of Impurities E and F present in the mixture is less than 100 ppm.

13. The process of claim 12 further comprising the step of determining the amount of Impurities E and F in the purified ridinilazole composition of step (b), and optionally in the crude ridinilazole composition of step (a).

14. The process of claim 13 wherein the determining step comprises HPLC-MS.

15. The process of any one of claims 12-14 wherein the crude ridinilazole composition of step (a) is provided by subjecting 3,3′-diaminobenzidine (DAB) to a condensation reaction to yield said ridinilazole.

16. The process of claim 15 wherein said condensation reaction comprises reacting DAB with an imidate.

17. The process of claim 16 wherein the imidate is methyl isonicotinimidate of formula (V):

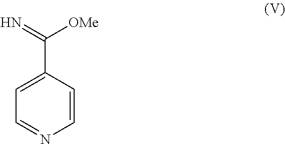

18. The process of any one of claims 15-17 wherein in step (a) said condensation reaction comprises:
(a) adding sodium methoxide to 4-cyanopyridine to produce the compound of formula (V); and then
(b) reacting the compound of formula (V) of step (a) with said DAB.

19. The process of claim 18 wherein in step (a) said condensation comprises:
(a) adding sodium methoxide to 4-cyanopyridine in methanol to produce the compound of formula (V); and then
(b) adding the compound of formula (V) of step (a) to a mixture of DAB and acetic acid in methanol; or
(c) adding a mixture of DAB and acetic acid in methanol to the compound of formula (V) of step (a).

20. The process of any one of claims 15-19 wherein said condensation reaction is carried out at a temperature of 20-90° C., for example 30-80° C., e.g. about 60° C.

21. The process of any one of claims 12-20 wherein the crude ridinilazole composition of step (a) is provided as set out in the following reaction scheme:

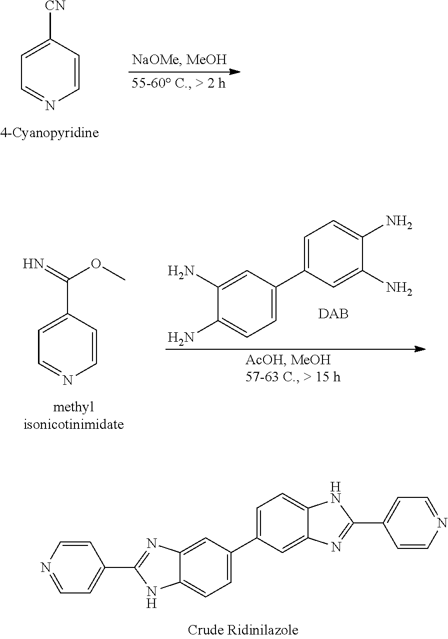

22. The process of any one of claims 16-21 wherein the compound of formula (II) is formed when DAB reacts with only one equivalent of the imidate, as shown below:

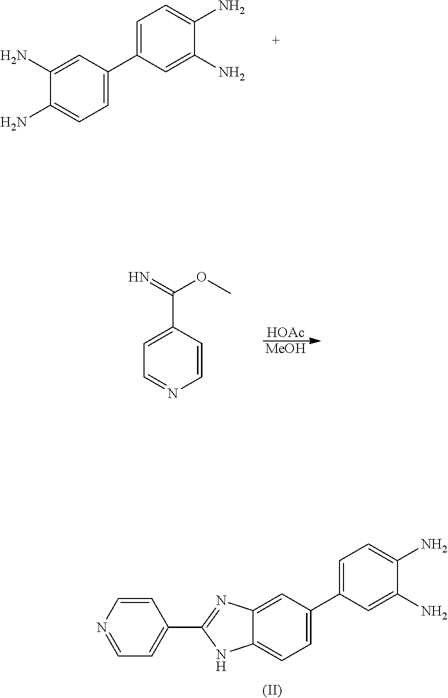

23. The process of any one of claims 17-22 wherein the compound of formula (IV) is formed by reaction of the methyl isonicotinimidate of formula (V) with monoaminobenzidine (MAB).

24. The process of claim 23 wherein the compound of formula (IV) is formed as set out in the following reaction scheme:

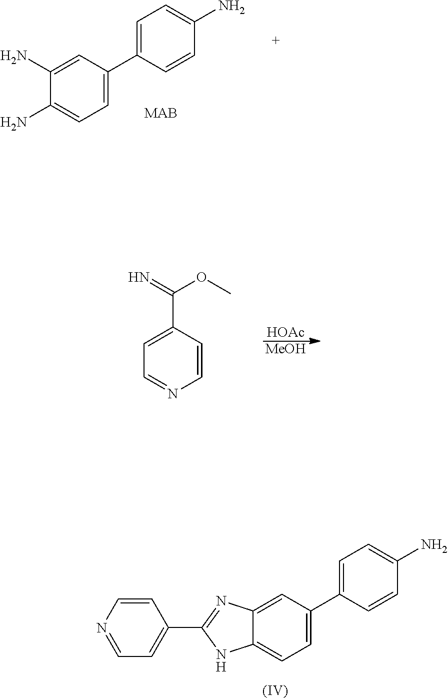

25. The process of any one of claims 12-24 wherein the removing step (b) produces a purified ridinilazole composition in which the amount of Impurity E present in the mixture is <50 ppm.

26. The process of any one of claims 12-25 wherein the removing step (b) produces a purified ridinilazole composition in which the amount of Impurity F present in the mixture is <50 ppm.

27. The process of any one of claims 12-26 wherein the removing step (b) produces a purified ridinilazole composition in which: (a) the amount of Impurity E present in the mixture is <50 ppm; and (b) the amount of Impurity F present in the mixture is <50 ppm.

28. The process of any one of claims 12-26 wherein the removing step (b) produces a purified ridinilazole composition in which the amount of Impurity E or that of Impurity F present in the mixture is greater than 50 ppm but less than 100 ppm.

29. The process of any one of claims 12-28 wherein the removing step (b) comprises treating the crude ridinilazole composition with an imidate solution, optionally wherein said imidate solution reacts with Impurity E and/or Impurity F to purge it/them from the mixture.

30. The process of any one of claims 12-29 wherein the removing step (b) comprises dissolving the crude ridinilazole composition and then reprecipitating the ridinilazole.

31. The process of claim 30 wherein the removing step (b) comprises forming a dissolved metal salt of the ridinilazole present in the crude ridinilazole composition and then precipitating ridinilazole, optionally by neutralization.

32. The process of claim 31 wherein the metal salt is an alkali metal salt, optionally selected from the sodium, potassium and lithium salts of ridinilazole.

33. The process of any one of claims 30-32 wherein the crude ridinilazole composition is dissolved with sodium methoxide in methanol, and then the ridinilazole is precipitated with acetic acid.

34. The process of any one of claims 12-33 wherein the removing step (b) comprises dissolving the crude ridinilazole composition in a high boiling aprotic solvent and then recrystallizing the ridinilazole.

35. The process of claim 34 wherein the high boiling aprotic solvent is DMSO.

36. The process of claim 34 or claim 35 wherein the removing step (b) further comprises slow cooling and/or temperature cycling of the solution.

37. The process of any one of claims 12-36 wherein the removing step (b) comprises solvent exchange with a ridinilazole alkali metal salt, optionally selected from the sodium, potassium and lithium salts of ridinilazole.

38. The process of any one of claims 12-37 wherein the removing step (b) comprises carbon treatment.

39. The process of claim 38 wherein the carbon treatment is applied to a solution of the crude ridinilazole mixture, optionally to an alkali metal ridinilazole salt solution, for example to a sodium, potassium or lithium ridinilazole salt solution.

40. The process of claim 39 wherein the carbon treatment comprises contact of the solution with activated carbon.

41. The process of claim 40 wherein the treatment with activated carbon further comprises the step of removing said activated carbon by filtration.

42. The process of any one of claims 38-41 wherein the carbon treatment comprises recirculation of the solution through an activated carbon filter cartridge.

43. The process of any one of claims 12-42 wherein the ridinilazole is present as:
(i) the anhydrous crystalline Form D, and the process comprises polymorph conversion from Form D to Form A; or
(ii) the crystalline form of ridinilazole tetrahydrate Form N, and the process comprises polymorph conversion from Form N to Form D; or
(iii) the crystalline form of ridinilazole tetrahydrate Form N, and the process comprises polymorph conversion from Form N to Form D, and then from Form D to Form A.

44. The process of claim 43 wherein the polymorph conversion comprises slurrying the crude ridinilazole composition in an aqueous solvent and then seeding the slurry with crystals of ridinilazole Form A at a water activity (A_{w}) and temperature favouring the crystallization of ridinilazole Form A.

45. The process of claim 44 wherein the A_{w} is ≥0.4 and/or the temperature is 2-60° C.

46. The process of claim 45 wherein the A_{w} is 0.4-0.5 and the temperature is >2° C. and <30° C.

47. The process of claim 46 wherein the A_{w} is 0.4-0.5 and the temperature is RT.

48. The process of any one of claims 44-47 wherein the solvent is MeOH/H₂O.

49. The process of any one of claims 44-48 wherein the Form A seeds: (a) are micronized; (b) in the form of a dry powder; or (c) in the form of a slurry.

50. The process of any one of claims 12-49 wherein the removing step (b) comprises:
(i) dissolving the crude ridinilazole composition and then reprecipitating the ridinilazole as defined in any one of claims 30-33;
(ii) dissolving the reprecipitated ridinilazole of step (i) and then recrystallizing the ridinilazole as defined in any one of claims 34-36;
(iii) subjecting the recrystallized ridinilazole of step (ii) to a carbon treatment as defined in any one of claims 38-42 to yield ridinilazole the anhydrous crystalline Form D characterized by an XRPD pattern substantially in accordance with FIG. 3; and
(iv) converting the Form D ridinilazole to Form A by polymorph conversion as defined in any one of claims 43-49.

51. A composition as defined in any one of claims 1-11 obtainable (or produced) by the process of any one of claims 12-50.

52. A pharmaceutical composition comprising an effective amount of the composition of any one of claim 1-11 or 51 and a pharmaceutically acceptable excipient.

53. The composition of any one of claims 1-11 and 51-52 for use in therapy or prophylaxis.

54. The composition of any one of claims 1-11 and 51-53 for use in the therapy or prophylaxis of CDI or CDAD.

55. Use of the composition of any one of claims 1-11 and 51-54 for the manufacture of a medicament for the treatment, therapy or prophylaxis of CDI or CDAD.

56. A crystalline form of ridinilazole tetrahydrate (Form A) characterized by a powder X-ray diffractogram comprising characteristic peaks at 2-Theta angles of (11.02±0.2)°, (16.53±0.2)° and (13.0±0.2)°.

57. The crystalline Form A of claim 56 characterized by an XRPD pattern substantially in accordance with FIG. 1.

58. The crystalline Form A of claim 56 or claim 57 which is substantially pure.

59. A composition comprising at least 80%, 90%, 95% or 99% w/w of the crystalline Form A of any one of claims 56-58.

60. The crystalline Form A of any one of claims 56-58 or composition of claim 59 in the form of micronized seed.

61. The crystalline Form A or composition of any one of claims 56-60 for use in the process of any one of claims 12-50, for example in a ridinilazole Form D to A polymorph conversion step as defined in any one of claims 43-49.

62. Use of the crystalline Form A or composition of any one of claims 56-60 in the manufacture of a composition as defined in any one of claims 1-11 and 51-54.

63. A crystalline form of ridinilazole anhydrate (Form D) characterized by a powder X-ray diffractogram comprising characteristic peaks at 2-Theta angles of (12.7±0.2)°, (23.18±0.2)° and (27.82±0.2)°, optionally comprising characteristic peaks at 2-Theta angles of (12.7±0.2)°, (23.18±0.2)°, (27.82±0.2)°, (19.5±0.2)° and (22.22±0.2)°.

64. The crystalline Form D of claim 63 characterized by an XRPD pattern substantially in accordance with FIG. 3.

65. The crystalline Form D of claim 63 or claim 64 which is substantially pure.

66. A composition comprising at least 80%, 90%, 95% or 99% w/w of the crystalline Form D of any one of claims 63-65.

67. The crystalline Form D or composition of any one of claims 63-66 for use in the process of any one of claims 12-50, for example in a ridinilazole Form D to A polymorph conversion step as defined in any one of claims 43-49.

68. Use of the crystalline Form D or composition of any one of claims 63-66 in the manufacture of a composition as defined in any one of claims 1-11 and 51-54.

69. Use of the crystalline Form D or composition of any one of claims 63-66 as an intermediate in the manufacture of a composition as defined in any one of claims 1-11 and 51-54.

70. A crystalline form of ridinilazole tetrahydrate (Form N) characterized by a powder X-ray diffractogram comprising characteristic peaks at 2-Theta angles of (10.82±0.2)°, (13.35±0.2)° and (19.15±0.2)°, optionally comprising characteristic peaks at 2-Theta angles of (10.82±0.2)°, (13.35±0.2)°, (19.15±0.2)°, (8.15±0.2)° and (21.74±0.2)°.

71. The crystalline Form N of claim 70 characterized by an XRPD pattern substantially in accordance with FIG. 2.

72. The crystalline Form N of claim 70 or claim 71 which is substantially pure.

73. A composition comprising at least 80%, 90%, 95% or 99% w/w of the crystalline Form N of any one of claims 70-72.

74. The crystalline Form N or composition of any one of claims 70-73 for use in the process of any one of claims 12-50, for example in a ridinilazole polymorph conversion step as defined in any one of claims 43-49.

75. Use of the crystalline Form N or composition of any one of claims 70-73 in the manufacture of a composition as defined in any one of claims 1-11 and 51-54.

76. Use of the crystalline Form N or composition of any one of claims 70-73 as an intermediate in the manufacture of a composition as defined in any one of claims 1-11 and 51-54.

77. The crystalline form, composition or use of any one of claims 56-76 wherein the XRPD is measured with Cu-Kalpha radiation having a wavelength of 0.15419 nm.

78. The crystalline form or composition of claim 77 wherein the XRPD is measured at room temperature.

79. An alkali metal salt of ridinilazole.

80. The alkali metal salt of claim 79 which is selected from the sodium, lithium and potassium salts of ridinilazole.

81. The alkali metal salt of claim 79 or claim 80 for use as an intermediate in the manufacture of a composition as defined in any one of claims 1-11 and 51-54.

82. Use of the alkali metal salt of any one of claims 79-81 as an intermediate in the manufacture of a composition as defined in any one of claims 1-11 and 51-54.
